(19) United States
(12) Patent Application Publication
(10) Pub. No.: US 2013/0150768 A1
(43) Pub. Date: Jun. 13, 2013

(54) Blood Purification Apparatus And Method For Inspecting Liquid Leakage Thereof

(71) Applicant: Nikkiso Company Limited, Tokyo (JP)

(72) Inventors: Kazuya SAKAMOTO, Makinohara-shi (JP); Tomohiro FURUHASHI, Makinohara-shi (JP)

(73) Assignee: Nikkiso Company Limited, Tokyo (JP)

(21) Appl. No.: 13/758,135

(22) Filed: Feb. 4, 2013

Related U.S. Application Data
(63) Continuation of application No. PCT/JP2011/067508, filed on Jul. 29, 2011.

(30) Foreign Application Priority Data
Aug. 5, 2010 (JP) 2010-176628

(51) Int. Cl. A61M 1/30; G01M 3/28

(52) U.S. Cl. CPC A61M 1/30; G01M 3/2846
USPC 604/6.09; 73/40.5 R

(57) ABSTRACT

A blood purification apparatus has a blood circuit, a blood pump and a blood purification instrument. A dialysate is introduced into and exits the blood purifying instrument. The blood purification apparatus has a pressure varying mechanism to vary pressure in a closed circuit under a condition where the blood circuit is formed as a closed circuit in a sealed condition connecting the tip end of the arterial blood circuit and the tip end of the venous blood circuit. A liquid leakage detecting mechanism detects liquid leakage in the blood circuit in accordance with pressure variation generated by the pressure varying mechanism.

### CROSS-REFERENCE TO RELATED APPLICATIONS

This application is a continuation of International Application No. PCT/JP2011/067508, filed Jul. 29, 2011, which claims priority to Japanese Application No. 2010-176628, filed Aug. 5, 2010. The disclosures of the above applications are incorporating herein by reference.

### FIELD

The present disclosure relates to a blood purification apparatus with a blood purification instrument (i.e. dialyzer) to purify blood of a patient by extracorporeally circulating blood in a blood circuit and a method for inspecting liquid leakage of the blood purification apparatus.

### BACKGROUND

In general, in blood purification apparatus used in dialysis treatment, it has a dialysate introducing line and a dialysate discharging line. The dialysate introducing line supplies the dialysate to the dialyzer. The dialysate discharge line discharges the dialysate, containing waste materials produced by dialysis, from the dialyzer. The dialysate introducing and discharge lines are connected to the dialyzer and to the blood circuit. Tip ends of the dialysate introducing line and the dialysate discharging line are connected, respectively, to a dialysate introducing port and a dialysate discharging port. Base ends of the lines are connected, respectively, to a dialysate supplying apparatus and a dialysate discharging apparatus.

Laid-open Japanese Patent Publication No. 253550/1999 describes technology of automatically detecting liquid leakage by applying a positive pressure to the blood circuit as well as dialysate lines of a main body side of the hemodialysis apparatus, including the dialysate introducing line and the dialysate discharging line. The liquid leakage detecting technology of this prior art is formed to detect the liquid leakage by closing a venous blood circuit at a portion near its tip end, using an electromagnetic valve etc. A blood pump, arranged in an arterial blood circuit, is driven to apply the positive pressure. It is determined if a pressure drop is detected.

However, according to the blood purification apparatus and the method for inspecting liquid leakage, the liquid leakage is detected by closing a venous blood circuit at a portion near its tip end using an electromagnetic valve etc. The positive pressure is applied by driving a blood pump arranged in a arterial blood circuit. Thus, it is impossible to perform liquid leakage inspection of a portion of the venous blood circuit positioned nearer its tip end, a portion closed by the electromagnetic valve, as well as a portion of the arterial blood circuit positioned nearer its tip end near the blood pump. In addition, since the liquid leakage inspection of the prior art is performed only by applying positive pressure, and the liquid leakage inspection due to application of the negative pressure is not performed, sufficient liquid leakage inspection cannot be achieved by the prior art.

### SUMMARY

It is, therefore, an object of the present disclosure to provide a blood purification apparatus and a method for inspecting liquid leakage that can perform a sufficient liquid leakage inspection over a whole region of the blood circuit.

To achieve the object, a blood purification apparatus comprises a blood circuit with an arterial blood circuit and a venous blood circuit. A blood pump is in the blood circuit to extracorporeally circulate blood of a patient. A blood purification instrument purifies the blood of a patient extracorporeally circulated through the blood circuit. The blood purification instrument is adapted to be connected with a base end of the arterial blood circuit and a base end of the venous blood circuit of the blood circuit. A dialysate introducing line introduces dialysate into the blood purifying instrument. A dialysate discharging line discharges the dialysate from the blood purifying instrument. The blood purification apparatus further comprises a pressure varying mechanism that varies the pressure in a closed circuit under a condition where the blood circuit is formed as a closed circuit in a sealed condition by connecting the tip end of the arterial blood circuit and the tip end of the venous blood circuit. A liquid leakage detecting mechanism is arranged in the closed circuit to detect liquid leakage in the blood circuit in accordance with pressure variation generated by the pressure varying mechanism.

The blood purification apparatus pressure varying mechanism varies liquid pressure in the closed circuit under a condition where the blood circuit is filled with priming liquid.

The blood purification apparatus pressure varying mechanism comprises a liquid supplying device that increases pressure in the closed circuit by applying a positive pressure to the closed circuit while introducing liquid into the closed circuit from the outside.

The blood purification apparatus further comprises a discharging line to discharge liquid or gas in the closed circuit to the outside. The liquid leakage in the blood circuit is detected by the liquid leakage detecting mechanism during a pressurizing step by alternately performing the pressurizing step and a priming step. In the pressurizing step, the positive pressure is applied to the closed circuit by introducing liquid into the closed circuit from the outside by the liquid supplying device while keeping the discharging line in a closed condition. In the priming step, liquid is introduced into the closed circuit from the outside by the liquid supplying device while keeping the discharging line in an opened condition. The liquid or gas in the closed circuit is discharged from the discharging line.

The blood purification apparatus liquid leakage detecting mechanism comprises a pressure detecting device to detect pressure in the closed circuit after a positive pressure has been applied to the closed circuit. A leak decision mechanism determines the existence of liquid leakage in the blood circuit based on the pressure detected by the pressure detecting device.

The blood purification apparatus pressure varying mechanism comprises a liquid supplying device to decrease pressure in the closed circuit by applying a negative pressure to the closed circuit while extracting liquid from the closed circuit to the outside.

The blood purification apparatus liquid leakage detecting mechanism comprises a pressure detecting mechanism to detect pressure in the closed circuit after a negative pressure has been applied to the closed circuit. A leak decision mechanism determines the existence of liquid leakage in the blood circuit based on the detected pressure by the pressure detecting mechanism.

The blood purification apparatus pressure detecting mechanism comprises one detector arranged in the closed circuit and the other detector arranged in the dialysate introducing line or the dialysate discharging line. The leak decision mechanism determines the existence of liquid leakage in the blood circuit by comparing a pressure detected by the pressure detecting detector arranged in the closed circuit with a pressure detected by the pressure detecting detector arranged in the dialysate introducing line or the dialysate discharging line.

The blood purification apparatus liquid leakage detecting mechanism comprises an air bubble detecting device to detect bubbles generated in the case of a liquid leakage in the blood circuit when the negative pressure is applied to the closed circuit. A leak decision mechanism determines the existence of liquid leakage based on whether air bubbles are detected.

The blood purification apparatus liquid supplying mechanism comprises a dialysate pump to introduce the dialysate into the blood purifying instrument, an ultrafiltration pump to perform ultrafiltration against blood circulating extracorporeally through the blood circuit, or a substitution infusing pump to introduce a substitution into the blood circuit.

The blood purification apparatus liquid supplying mechanism is a pump able to perform a normal rotation and a reverse rotation. Either the application of positive pressure or negative pressure to the closed circuit can be achieved by selectively performing the normal rotation or the reverse rotation.

The blood purification apparatus pressure varying mechanism is able to increase pressure in the closed circuit by applying a positive pressure to the closed circuit while introducing liquid into the closed circuit from the outside. Also, it is able to decrease pressure in the closed circuit by applying a negative pressure to the closed circuit while extracting liquid from the closed circuit to the outside. The liquid leakage detecting mechanism is able to detect liquid leakage in both cases of the application of the positive pressure and negative pressure.

In the blood purification apparatus, the positive pressure is applied to the closed circuit after the negative pressure has been applied to the closed circuit.

The blood purification apparatus pressure varying mechanism varies pressure in the closed circuit by introducing liquid into the closed circuit through the dialysate introducing line or by discharging liquid from the closed circuit to the dialysate discharging line. The liquid leakage detecting mechanism is able to detect liquid leakage in the dialysate introducing line or the dialysate discharging line in addition to liquid leakage in the closed circuit.

A blood purification apparatus includes a blood circuit with an arterial blood circuit with a blood pump and a venous blood circuit. The blood circuit extracorporeally circulates blood of a patient via a blood pump. A blood purification instrument purifies the blood of a patient by extracorporeally circulating the blood through the blood circuit. The blood purification instrument is adapted to be connected with a base end of the arterial blood circuit and a base end of the venous blood circuit of the blood circuit. A dialysate introducing line introduces dialysate into the blood purifying instrument. A dialysate discharging line discharges the dialysate from the blood purifying instrument. A method comprises the steps of varying pressure in a closed circuit under a condition where the blood circuit is formed as a closed circuit in a sealed condition by connecting the tip end of the arterial blood circuit and the tip end of the venous blood circuit. A liquid leakage detecting step, to detect liquid leakage in the blood circuit, is conducted in accordance with pressure variation generated by the pressure varying step.

The method for inspecting liquid leakage of a blood purification apparatus wherein during the pressure varying step, the liquid pressure in the closed circuit is varied under a condition where the blood circuit is filled with priming liquid.

The method for inspecting liquid leakage of a blood purification apparatus wherein during the pressure varying step, the pressure in the closed circuit is increased by applying a positive pressure to the closed circuit while introducing liquid into the closed circuit from the outside.

The method for inspecting liquid leakage of a blood purification apparatus wherein the blood purification apparatus further comprises a discharging line to discharge liquid or gas in the closed circuit to the outside. The liquid leakage detecting step is performed during a pressurizing step by alternately performing the pressurizing step and a priming step. In the pressurizing step, the positive pressure is applied to the closed circuit by introducing liquid into the closed circuit from the outside by the liquid supplying mechanism while keeping a closed condition of the discharging line. In the priming step, liquid is introduced into the closed circuit from the outside by the liquid supplying mechanism while keeping an opened condition of the discharging line and then liquid or gas in the closed circuit is discharged from the discharging line.

The method for inspecting liquid leakage of a blood purification apparatus wherein the liquid leakage detecting step comprises a pressure detecting step for detecting pressure in the closed circuit after a positive pressure has been applied to the closed circuit. Also, a decision step is performed to determine the existence of liquid leakage in the blood circuit on the basis of the pressure detected in the pressure detecting step.

The method for inspecting liquid leakage of a blood purification apparatus wherein in the pressure varying step, the pressure in the closed circuit is decreased by applying a negative pressure to the closed circuit while extracting liquid from the closed circuit to the outside.

The method for inspecting liquid leakage of a blood purification apparatus wherein the liquid leakage detecting step comprises a pressure detecting step for detecting pressure in the closed circuit after a negative pressure has been applied to the closed circuit. Also, a decision step is performed to determine the existence of liquid leakage in the blood circuit on the basis of pressure detected in the pressure detecting step.

The method for inspecting liquid leakage of a blood purification apparatus wherein in the pressure detecting step, pressures in the closed circuit and in the dialysate introducing line or the dialysate discharging line are detected, respectively. The liquid leakage in the blood circuit is determined by comparing a pressure detected in the closed circuit with a pressure detected in the dialysate introducing line or the dialysate discharging line.

The method for inspecting liquid leakage of a blood purification apparatus wherein the liquid leakage detecting step comprises an air bubble detecting step for detecting bubbles generated in the case of liquid leakage in the blood circuit when the negative pressure is applied to the closed circuit. Also, a decision step is performed to determine the existence of liquid leakage based on whether the air bubbles are detected.

The method for inspecting liquid leakage of a blood purification apparatus wherein the introduction or discharge of liquid to or from the closed circuit in the pressure varying step is performed by a dialysate pump to introduce the dialysate into the blood purifying instrument; an ultrafiltration pump to perform ultrafiltration against blood circulating extracorporeally through the blood circuit; or a substitution infusing pump to introduce a substitution into the blood circuit.

The method for inspecting liquid leakage of a blood purification apparatus wherein the introduction or discharge of liquid to or from the closed circuit in the pressure varying step is achieved by a pump being able to perform a normal rotation and a reverse rotation. The application of positive pressure or negative pressure to the closed circuit can be achieved by selectively performing the normal rotation or the reverse rotation.

The method for inspecting liquid leakage of a blood purification apparatus wherein in the pressure varying step, the pressure in the closed circuit is increased by applying a positive pressure to the closed circuit while introducing liquid into the closed circuit from the outside. Also, pressure is decreased by applying a negative pressure to the closed circuit while extracting liquid from the closed circuit to the outside. The liquid leakage detection is performed in both cases by the application of the positive pressure and negative pressure.

The method for inspecting liquid leakage of a blood purification apparatus wherein the positive pressure is applied to the closed circuit after the negative pressure has been applied to the closed circuit.

The method for inspecting liquid leakage of a blood purification apparatus wherein in the pressure varying step, the pressure in the closed circuit is varied by introducing liquid into the closed circuit through the dialysate introducing line or by discharging liquid from the closed circuit into the dialysate discharging line. The liquid leakage detecting step, the liquid leakage in the dialysate introducing line or the dialysate discharging line is detected in addition to liquid leakage in the closed circuit.

Liquid leakage in the blood circuit is detected by varying pressure in a closed circuit formed by connecting a tip end of the arterial blood circuit and a tip end of the venous blood circuit. Thus, it is possible to perform a sufficient liquid leakage inspection over a whole region of the blood circuit.

Liquid leakage in the blood circuit is detected by varying the liquid pressure in the closed circuit under a condition where the blood circuit is filled with priming liquid. Thus, it is possible to perform a sufficient liquid leakage inspection over a whole region of the blood circuit.

Pressure variation in the closed circuit is performed by increasing the pressure while applying a positive pressure to the closed circuit by introducing liquid into the closed circuit from the outside. Thus, it is possible to prevent the sucking in of air into the blood circuit in the case of the liquid leakage and thus it is possible to smoothly and properly perform the blood purification treatment.

The blood purification apparatus further comprises a discharging line to discharge liquid or gas in the closed circuit to the outside. The liquid leakage in the blood circuit is detected during a pressurizing step by alternately performing the pressurizing step and a priming step. In the pressurizing step, the positive pressure is applied to the closed circuit by introducing liquid into the closed circuit from the outside by the liquid supplying mechanism while keeping the discharging line in a closed condition. In the priming step, liquid is introduced into the closed circuit from the outside by the liquid supplying mechanism while keeping the discharging line in an opened condition. Liquid or gas in the closed circuit is discharged from the discharging line. Thus, it is possible to perform the detection of liquid leakage during a process where the priming (discharge of air bubbles and filling of priming liquid) is performed.

The liquid leakage detection is performed by detecting pressure in the closed circuit after a positive pressure has been applied to the closed circuit and by determining the existence of liquid leakage in the blood circuit on the basis of the detected pressure. Thus, it is possible to detect the pressure when the positive pressure is applied to the closed circuit for example by substituting a venous pressure sensor. The venous pressure sensor is usually connected to an air trap chamber connected to the venous blood circuit as a positive pressure detecting sensor. Thus, this reduces the manufacturing cost of the blood purification apparatus.

The pressure varying step is performed by applying a negative pressure to the closed circuit while extracting liquid from the closed circuit to the outside to decrease the pressure in the closed circuit. Thus, it is possible to achieve the liquid leakage test due to the negative pressure of a portion where the negative pressure is applied (e.g. a portion nearer to the tip end side of the arterial blood circuit than the blood pump) during the blood purification treatment. Also, it is possible to achieve the liquid leakage test proper for actions applied during the blood purification treatment.

The liquid leakage detection is performed by detecting pressure in the closed circuit after a negative pressure has been applied to the closed circuit, and by determining the existence of liquid leakage in the blood circuit on the basis of the detected pressure. Thus, it is possible to detect the pressure when the negative pressure is applied to the closed circuit for example by substituting a venous pressure sensor. The venous pressure sensor is usually connected to an air trap chamber connected to the venous blood circuit as a negative pressure detecting sensor. Thus, this reduces the manufacturing cost of the blood purification apparatus.

The pressure detection is performed respectively in the closed circuit, the dialysate introducing line or the dialysate discharging line. The existence of the liquid leakage is determined by comparing a pressure detected in the closed circuit with a pressure detected in the dialysate introducing line or the dialysate discharging line. Thus, it is possible to further improve the accuracy of determining the liquid leakage.

The detection of liquid leakage is performed by detecting bubbles generated in a case of liquid leakage in the blood circuit when the negative pressure is applied to the closed circuit. Thus, it is possible to detect the bubbles even when the negative pressure is applied to the closed circuit by substituting a bubble detecting sensor. The bubble detecting sensor is usually connected to the arterial blood circuit at a position nearer to the tip end than the air trap chamber. Thus, it is possible to reduce the manufacturing cost of the blood purification apparatus. In addition, since bubbles are generated in the case of liquid leakage, the liquid leakage can be detected by visual observation.

The introduction and discharge of liquid to or from the closed circuit is performed by using the dialysate pump to introduce dialysate to the blood purification instrument; an ultrafiltration pump to perform ultrafiltration against blood extracorporeally circulating through the blood circuit; or the substitution infusing pump. Thus, it is possible to substitute pumps used in the blood purification treatment for these pumps. Thus, this also reduces the manufacturing cost of the blood purification apparatus.

The introduction or discharge of liquid to or from the closed circuit in the pressure varying step is achieved by a pump being able to perform a normal rotation and a reverse rotation. The application of positive pressure or negative pressure into the closed circuit can be achieved by selectively performing the normal rotation or the reverse rotation. Thus, it is possible to arbitrarily and easily perform the application of positive pressure or negative pressure.

In the pressure varying step of the closed circuit, the pressure in the closed circuit is increased by applying a positive pressure into the closed circuit while introducing liquid into the closed circuit from the outside. Also, pressure is decreased by applying a negative pressure to the closed circuit while extracting liquid from the closed circuit to the outside. Thus, the liquid leakage detection is performed in both cases of the application of the positive pressure and negative pressure. Accordingly, it is possible to perform a proper and sufficient liquid leakage inspection.

The positive pressure is applied to the closed circuit after the negative pressure has been applied to the closed circuit. Thus, it is possible to prevent sucking in of air into the blood circuit through both tip ends of the arterial blood circuit and the venous blood circuit and thus to smoothly perform the blood purification treatment.

The liquid leakage in the dialysate introducing line or the dialysate discharging line can be detected in addition to liquid leakage in the closed circuit. Thus, it is possible to further improve the reliability of the blood purification apparatus.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a schematic view of a hemodialysis apparatus (before formation of a closed circuit) of a first embodiment.

FIG. 2 is a schematic view of a condition of the positive pressure application to the closed circuit in the hemodialysis apparatus of the first embodiment.

FIG. 3 is a schematic view of a condition of the negative pressure application to the closed circuit in the blood purification apparatus of the first embodiment.

FIG. 4 is a schematic view of a condition of the positive pressure application to the closed circuit in the blood purification apparatus of the first embodiment.

FIG. 5 is a schematic view of a condition of the positive pressure application to the closed circuit in the hemodialysis apparatus of a second embodiment.

FIG. 6 is a schematic view of a condition of the negative pressure application to the closed circuit in the blood purification apparatus of the second embodiment.

FIG. 7 is a schematic view of a condition of the positive pressure application to the closed circuit in the blood purification apparatus of the second embodiment.

FIG. 8 is a schematic view of a condition of the positive pressure application to the closed circuit in the hemodialysis apparatus of a third embodiment.

FIG. 9 is a schematic view of a condition of the negative pressure application to the closed circuit in the blood purification apparatus of the third embodiment.

FIG. 10 is a schematic view of a condition of a pressure applying step in the blood purification apparatus of a fourth embodiment.

FIG. 11 is a schematic view of a condition of a liquid leakage detection in the blood purification apparatus of the fourth embodiment.

FIG. 12 is a schematic view of a condition of a priming step in the blood purification apparatus of the fourth embodiment.

FIG. 13 is a schematic view of a condition of a circulation step in the blood purification apparatus of the fourth embodiment.

FIG. 14 is a schematic view of an embodiment where pressure detecting devices are connected both to the closed circuit and the dialysate introducing line of the blood purification apparatus.

FIG. 15 is a schematic view of an embodiment where pressure detecting mechanisms are connected to both the closed circuit and the dialysate introducing line of the blood purification apparatus.

FIG. 16 is a schematic view of an embodiment where pressure detecting mechanisms are connected to both the closed circuit and the dialysate introducing line of the blood purification apparatus.

FIG. 17 is a schematic view of an embodiment where pressure detecting mechanism is connected to both the closed circuit and the dialysate introducing line of the blood purification apparatus.

FIG. 18 is a schematic view of a condition of the positive or negative pressure application to the closed circuit in the hemodialysis apparatus of another embodiment of the present disclosure; and

FIG. 19 is a schematic view of a condition of the positive pressure application to the closed circuit in the hemodialysis apparatus of a further embodiment.

### DETAILED DESCRIPTION

Preferable embodiments of the present disclosure will be hereinafter described with reference to the drawings.

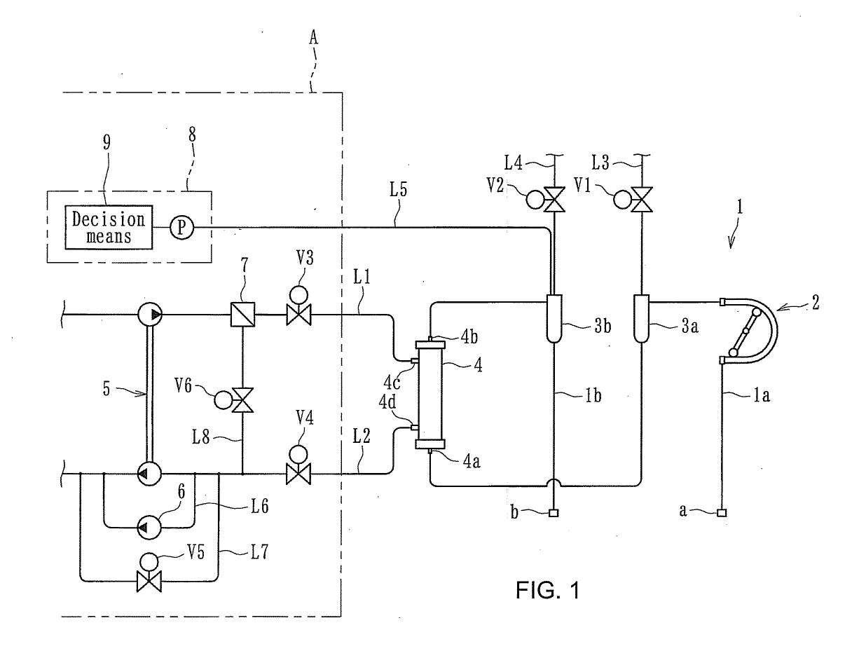

A blood purification apparatus of a first embodiment is adapted to be applied to a hemodialysis apparatus and includes, as shown in FIGS. 1-4, a blood circuit 1 including an arterial blood circuit 1a and a venous blood circuit 1b. A dialyzer 4 functions as a blood purification instrument. A dialysate introducing line L1 and a dialysate discharging line L2 are coupled with the dialyzer. A duplex pump 5 and an ultrafiltration pump 6 functions as a liquid supplying mechanism forming a pressure varying device. A liquid leakage detecting mechanism 8 is shown. The duplex pump 5 also functions as a dialysate pump and a pump for introducing the dialysate into the dialyzer 4. A character “A” in the drawings denotes a main body of the hemodialysis apparatus.

The dialyzer 4 is intended to purify extracorporeally circulating blood of a patient. It contains membranes (not shown), such as hollow fiber membranes, semi-permeable membranes and filtration membranes. The dialyzer 4 is provided with a blood introducing port 4a, to introduce blood, and a blood discharging port 4b, to discharge the introduced blood. A dialysate introducing port 4c introduces dialysate and is connected with a tip end of the dialysate introducing line L1. A dialysate discharging port 4d discharges the introduced dialysate. The discharging port 4d is connected with a tip end of the dialysate discharging line L2. Thus, as is well known in the art, the blood purification action can be performed by contacting the dialysate with the blood introduced through the blood introducing port 4a via the hollow fiber membranes.

The blood introducing port 4a is connected with a base end of the arterial blood circuit 1a. A blood pump 2 and an arterial air trap chamber 3a are arranged on the arterial blood circuit 1a. The blood discharging port 4b is connected with a base end of the venous blood circuit 1b. A venous air trap chamber 3b is arranged on the venous blood circuit 16. A connector “a” and a connector “b” are arranged on the tip ends, respectively, of the arterial blood circuit 1a and the venous blood circuit 1b. The connectors “a” and “b” are adapted to be attached, respectively, with an arterial puncture needle and a venous puncture needle.

Thus, it is possible to extracorporeally circulate blood of a patient while puncturing the arterial puncture needle and the venous puncture needle into an artery and a vein of a patient and driving the blood pump 2. During the extracorporeal circulation of the blood of a patient, bubble removal is performed through the arterial air trap chamber 3a, connected to the arterial blood circuit 1a, and the venous air trap, chamber 3b, connected to the venous blood circuit 1b. The blood pump 2 is formed by a peristaltic pump. The peristaltic pump is able to feed blood from the arterial puncture needle toward the blood introducing port 4a of the dialyzer 4. The pump 2 squeezes a flexible tube, forming the arterial blood circuit 1a, by normally rotating the pump.

Overflow lines L3, L4 are connected to top end portions (air layer), respectively, of the arterial air trap chamber 3a and venous air trap chamber 3b. Electromagnetic valves V1, V2 are also arranged, respectively, on the overflow lines L3, L4. Thus, it is possible to arbitrarily open and close the flow paths of the overflow lines L3, L4 by opening and closing the electromagnetic valves V1, V2. Accordingly, it is possible to form a sealed condition of the blood circuit 1 by connecting the tip ends of the arterial blood circuit 1a and the venous blood circuit 1b and by closing the electromagnetic valves V1, V2.

In addition, a liquid pressure monitoring line L5 is extended from the top end portion (air layer) of the venous air trap chamber 3b. The tip end of the liquid pressure monitoring line L5 is connected to a venous pressure sensor P arranged within the main body “A” of the hemodialysis apparatus. The venous pressure sensor P is able to detect the liquid pressure in the blood circuit 1, more particularly the venous blood circuit 1b, by detecting the pressure in the top portion (air layer) of the venous air trap chamber 3b. Thus, it is possible to continuously (in real time) detect the venous pressure during the blood purification treatment (hemodialysis treatment).

Base ends of the dialysate introducing line Li and the dialysate discharging line L2 are connected, respectively, to a dialysate supplying apparatus (dialysate supplying source, not shown) and a dialysate discharging apparatus (also not shown). The duplex pump (liquid supplying mechanism) 5 is arranged across the dialysate introducing line Li and the dialysate discharging line L2. By operating the duplex pump 5, the prepared dialysate is supplied to the dialyzer 4 from the dialysate supplying apparatus through the dialysate introducing line L1. The used dialysate is discharged from the dialyzer 4 and returned to the dialysate discharging apparatus through the dialysate discharging line L2.

Bypass line L6, L7 bypassing the duplex pump 5, are provided on the dialysate discharging line L2. An ultrafiltration pump (liquid supplying mechanism) 6, for removing water content from blood of a patient flowing through the dialyzer 4, is arranged on the bypass line L6. An electromagnetic valve V5, for opening and closing a flow path of the bypass line L7, is arranged on it. In addition, an electromagnetic valve V4, for opening and closing a flow path of the dialysate discharging line L2, is arranged on the dialysate discharging line L2 at a position near the dialysate discharging port 4d of the dialyzer 4.

A filter 7 is arranged on the dialysate introducing line L1. An electromagnetic valve V3, for opening and closing a flow path of the dialysate introducing line L1, is arranged on the dialysate introducing line L1 at a position near the dialysate introducing port 4c of the dialyzer 4. The filter 7 is intended to filtrate and purify the dialysate flowing through the dialysate introducing line L1. The filter 7 includes a primary chamber where dialysate, to be filtrated, flows through a filtering membrane. A secondary chamber in the filter is where the filtrated dialysate flows. In addition, a bypass line L8 extends from the filter 7 for bypassing dialysate to the dialysate discharging line L2. An electromagnetic valve V6, for opening and closing a flow path of the bypass line L8, is arranged on the bypass line L8.

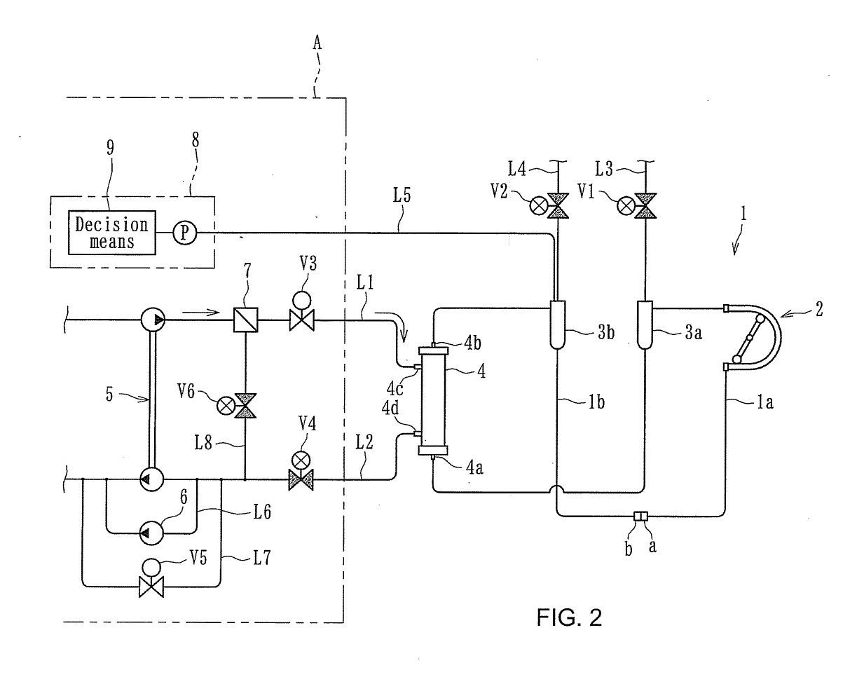

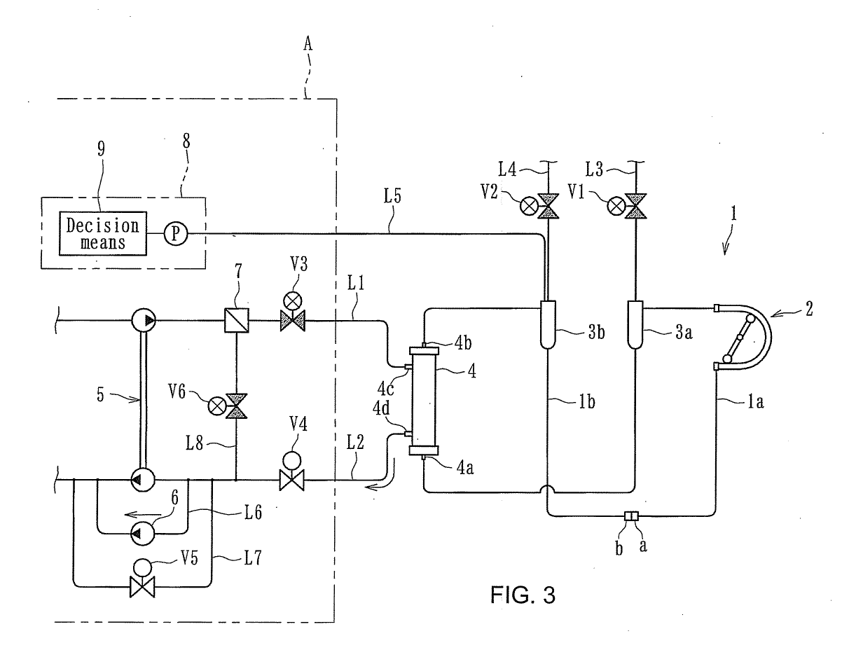

As shown in FIGS. 2 and 3, the blood purification apparatus of this embodiment has a pressure varying mechanism for varying pressure in a closed circuit under a condition where the blood circuit 1 is formed as a closed circuit in a sealed condition connecting the tip end (connector) “a” of the arterial blood circuit is and the tip end (connector) “b” of the venous blood circuit 1b. The blood circuit 1 is filled with priming liquid. A liquid leakage detecting mechanism 8 is arranged in the closed circuit to detect liquid leakage in the blood circuit 1 in accordance with pressure variation generated by the pressure varying mechanism.

The pressure varying mechanism of the present disclosure includes the duplex pump (liquid supplying mechanism) 5 and the ultrafiltration pump 6. The duplex pump 5 increases liquid pressure in the closed circuit by applying a positive pressure to the closed circuit while introducing liquid (dialysate) into the closed circuit from the outside (dialysate introducing line L1) of the closed circuit. The ultrafiltration pump (liquid supplying mechanism) 6 decreases liquid pressure in the closed circuit by applying a negative pressure to the closed circuit while extracting liquid (priming solution (dialysate) from the closed circuit to the outside (dialysate discharging line L2). The pressure varying mechanism is constructed so that it is possible to increase the liquid pressure in the closed circuit by applying a positive pressure to the closed circuit while introducing liquid (dialysate) into the closed circuit from the outside using the duplex pump 5. Also, it is possible to decrease the liquid pressure in the closed circuit by applying a negative pressure to the closed circuit while extracting liquid (priming solution) from the closed circuit to the outside using the ultrafiltration pump 6. Thus, it is possible to perform the liquid leakage detection using the liquid leakage detecting mechanism 8 in the application of both the positive pressure and the negative pressure.

As described above, it is possible to apply the positive pressure to the closed circuit by forming the closed circuit in a sealed condition. Here, the blood circuit 1 is closed by closing the electromagnetic valves V1, V2 and accordingly the overflow lines L3, L4. Driving of the duplex pump (liquid supplying mechanism) 5 with closing of the electromagnetic valves V4, V6 and opening other electromagnetic valves as shown in FIG. 2 enable priming of the blood circuit 1 with the priming solution (e.g. dialysate or physiological saline) with the tip end (connector “a”) of the arterial blood circuit 1a and the tip end (connector “b”) of the venous blood circuit 1b in the connected condition. Thus, by introducing (i.e. inverse filtrating), the dialysate in the dialysate introducing line L1 (portion between the duplex pump 5 and the dialyzer 4) and the dialysate discharging line L2 (portion between the dialyzer 4 and the electromagnetic valve V4) from the flow path of dialysate to the blood flow path through the filtration membrane (hollow fiber membrane in the present embodiment) of the dialyzer 4, the positive pressure is created.

On the contrary, it is possible to apply the negative pressure to the closed circuit by forming the closed circuit in a sealed condition in the blood circuit 1. This is accomplished by closing the electromagnetic valves V1, V2 and accordingly the overflow lines L3, L4 and normally rotating the ultrafiltration pump (liquid supplying mechanism) 6 while closing the electromagnetic valves V3, V6 and opening other electromagnetic valves as shown in FIG. 3. After the priming has been performed, the blood circuit 1 is filled with priming solution (e.g. dialysate or physiological saline) with the tip end (connector “a”) of the arterial blood circuit 1a and the tip end (connector “b”) of the venous blood circuit 1b in the connected condition. Thus, by extracting (i.e. normally filtrating) the priming solution in the closed circuit to the dialysate discharging line L2 through the filtration membrane (hollow fiber membrane in the present embodiment) of the dialyzer 4, the negative pressure is created.

The liquid leakage detecting device 8 includes the venous pressure sensor P and a decision mechanism 9. As previously described, the venous pressure sensor (liquid pressure detecting sensor) P is able to continuously (in real time) detect the venous pressure during the blood purification treatment (hemodialysis treatment). Additionally, it is able to continuously (in real time) detect the liquid pressure after the positive pressure has been applied to the closed circuit by the duplex pump 5 and after the negative pressure has been applied to the closed circuit by the ultrafiltration pump 6.

The decision mechanism 9 is formed by e.g. a microcomputer etc. arranged within the main body “A” of the hemodialysis apparatus. The decision mechanism 9 is electrically connected to the venous pressure sensor (liquid pressure detecting device) P to determine the existence of liquid leakage in the blood circuit 1 on the basis of the liquid pressure detected by the venous pressure detecting sensor P. That is, the existence of liquid leakage is decided by the decision mechanism 9 in accordance with tendency after variation of the detected liquid pressure with continuously (in real time) detecting the tendency after variation of the liquid pressure caused by the pressure varying mechanism using the venous pressure sensor P. There are methods for determining the liquid leakage using such a decision mechanism 9.

First, the pressure varying mechanism applies a positive or negative pressure to the closed circuit and keeps a constant liquid pressure. The venous pressure sensor (liquid pressure detecting device) P detects a liquid pressure having been varied until a predetermined period has elapsed from the constantly kept liquid pressure. Finally, a determination is made whether the detected liquid pressure exceeds a predetermined threshold value (upper limit or lower limit value). When the detected liquid pressure exceeds the threshold, since it is supposed that it is impossible to keep the constant liquid pressure due to the liquid leakage, it can be determined that the liquid leakage would exist in any portion of the closed circuit. In another method for determining the liquid leakage using such a decision mechanism 9, the pressure varying mechanism applies a positive or negative pressure to the closed circuit. The venous pressure sensor (liquid pressure detecting sensor) P detects the liquid pressure and determines whether the detected liquid pressure is a predetermined liquid pressure. When the detected liquid pressure does not exhibit the predetermined liquid pressure, since it is supposed that the liquid pressure cannot reach the predetermined value due to the liquid leakage, it can be determined that the liquid leakage would exist in any portion of the closed circuit.

The method for inspecting liquid leakage of a dialysis apparatus of the first embodiment will be described.

First, the liquid pressure in the closed circuit is varied by the pressure varying mechanism under a condition where the blood circuit 1 is formed as a closed circuit in a sealed condition connecting the tip end of the arterial blood circuit 1a and the tip end of the venous blood circuit 1b. Also, it is under a condition where the blood circuit 1 is filled with the priming solution (pressure varying step). The liquid leakage in the blood circuit 1 is detected by the liquid leakage detecting mechanism in accordance with the variation of the liquid pressure in the pressure varying step (liquid leakage detecting step).

The liquid leakage detecting step includes a pressure detecting step to detect liquid pressure in the closed circuit after a positive pressure or a negative pressure has been applied to the closed circuit. A decision step determines the existence of liquid leakage in the blood circuit 1 based on the liquid pressure detected in the pressure detecting step. In addition, in the pressure varying step, the liquid pressure in the closed circuit is increased by applying a positive pressure to the closed circuit by driving the duplex pump 5 while introducing liquid into the closed circuit from the outside. Also, the pressure is decreased by applying a negative pressure to the closed circuit by normally rotating the ultrafiltration pump 6 while extracting liquid from the closed circuit to the outside. When the liquid leakage is detected in the liquid leakage detecting step, the fact is informed to an operator to urge him, confirming the liquid leakage.

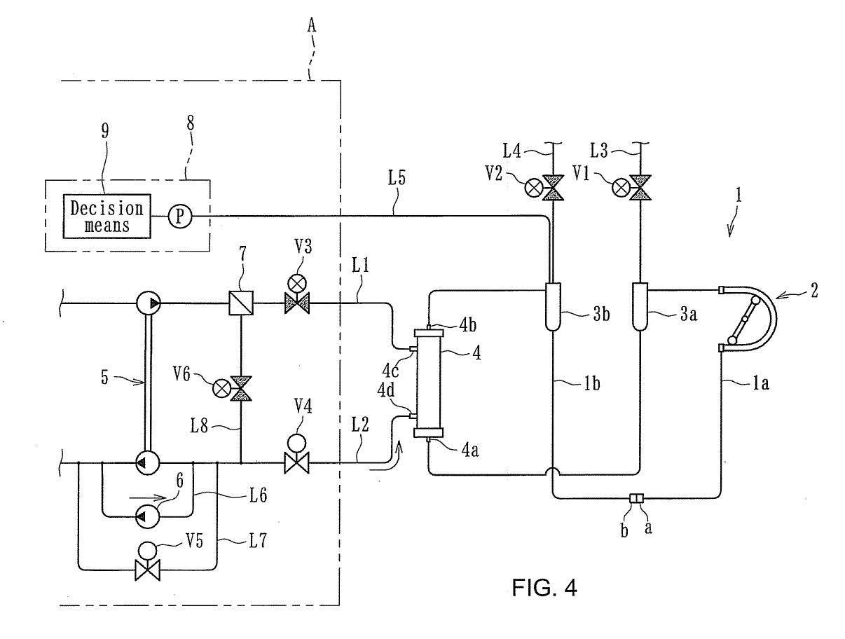

It is preferable that the ultrafiltration pump 6, functioning as the liquid supplying mechanism, is a pump able to rotate both in normal and reverse directions so that either a positive pressure or a negative pressure can be applied to the closed circuit by selectively driving the ultrafiltration pump 6 in the normal direction or reverse direction. That is, as previously described, when the ultrafiltration pump 6 is normally rotated, it is possible to apply a negative pressure to the closed circuit. On the contrary, as shown in FIG. 4, when the ultrafiltration pump 6 is reversely rotated, the dialysate in the dialysate discharging line L2 is introduced into the flow path of the blood from the flow path of the dialysate of the dialyzer 4 through the filtration membrane (hollow fiber membrane in the present embodiment) (inverse filtration). Thus, the positive pressure can be applied to the closed circuit. According to such a construction, it is possible to easily and arbitrary apply the positive pressure and negative pressure to the closed circuit.

As described above, the pressure varying mechanism and the pressure varying step can vary the liquid pressure in the closed circuit by introducing liquid into the closed circuit from the dialysate introducing line L1 or by extracting liquid from the closed circuit into the dialysate discharging line L2. The liquid leakage detecting mechanism and the liquid leakage detecting step can detect the liquid leakage of the dialysate introducing line L1 or the dialysate discharging line L2. Liquid leakage can be detected in a positive pressure applying region of the dialysate introducing line L1 or in a negative pressure applying region of the dialysate discharging line L2 in addition to the closed circuit. Accordingly, since it is possible to detect liquid leakage of the dialysate introducing line L1 or the dialysate discharging line L2 in addition to the closed circuit, the liquid leakage in a wide region can be achieved. Thus, it is possible to improve the reliability of the blood purification apparatus.

According to this embodiment and also the same in following embodiment, it is possible to detect liquid leakage at connections between the blood circuit 1 and other structural elements or liquid flow paths forming the blood purification apparatus, or at connections between the dialysate introducing line L1 or the dialysate discharging line L2 and other structural elements or liquid flow paths forming the blood purification apparatus. For example, it is possible to detect liquid leakages at connections between the dialysate introducing port 4c and dialysate introducing line L1; between the dialysate discharging port 4d and the dialysate discharging line L2; connections between the blood introducing port 4a of the dialyzer 4 and arterial blood circuit 1a; and between blood discharging port 4b and the venous blood circuit 1b.

In the first embodiment, although it is described that the pressure varying step is performed under the condition where the priming solution is filled in the blood circuit 1, it may be performed under a condition where air, before the priming, is filled in the blood circuit 1. In this case, the liquid leakage is detected so that the pressure varying mechanism varies atmosphere (pressure) in the closed circuit and the liquid leakage detecting mechanism detects the liquid leakage in accordance with the atmosphere variation.

A blood purification apparatus of a second embodiment will be described.

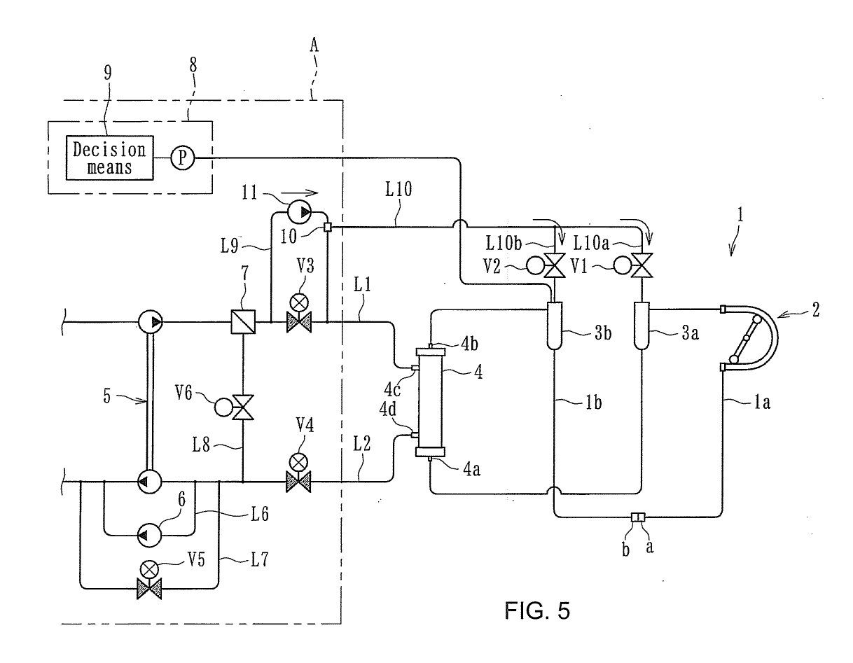

Similar to the first embodiment, the blood purification apparatus of this embodiment is adapted to be applied to a hemodialysis apparatus. It mainly includes, as shown in FIGS. 5-7, a blood circuit 1 with an arterial blood circuit 1a, a venous blood circuit 1b, a dialyzer 4 functioning as a blood purification instrument, a dialysate introducing line L1, a dialysate discharging line L2, a duplex pump 5, an ultrafiltration pump 6 functioning as the liquid supplying mechanism forming a pressure varying mechanism, and a liquid leakage detecting mechanism 8. The same reference numerals will also be used to designate the same structural elements in the first embodiment and their detailed description will be omitted.

A bypass line L9, bypassing the electromagnetic valve 3, is connected to the dialysate introducing line L1. A substitution infusing pump 11 and a substitution infusing port 10 are arranged on the way of the bypass line L9. The base end of a substitution infusing line L10 is connected to the substitution infusing port 10. The tip end of the substitution infusing line L10 is connected to the top portions (air layer) of the arterial air trap chamber 3a and the venous air trap chamber 3b, via a branch line L10a and a branch line L10b, respectively. The substitution infusing pump 11 is a displacement type pump and adapted to introduce dialysate into the blood circuit 1 during the blood purification treatment.

When driving the substitution infusing pump 11 during the blood purification treatment, the dialysate flowing through the dialysate introducing line L1 is introduced into the blood circuit 1 via the bypass line L9, the substitution infusing line L10 and the branch lines L10a, L10b. Thus, the substitution infusion is performed. For performing the pre-dialysate during the blood purification treatment, the dialysate, functioning as substitution, is introduced into the arterial blood circuit 1a by opening the electromagnetic valve V1 and closing the electromagnetic valve V2. To perform the post-dialysate during the blood purification treatment, the dialysate, functioning as the substitution, is introduced into the venous blood circuit 1b by closing the electromagnetic valve V1 and opening the electromagnetic valve V2.

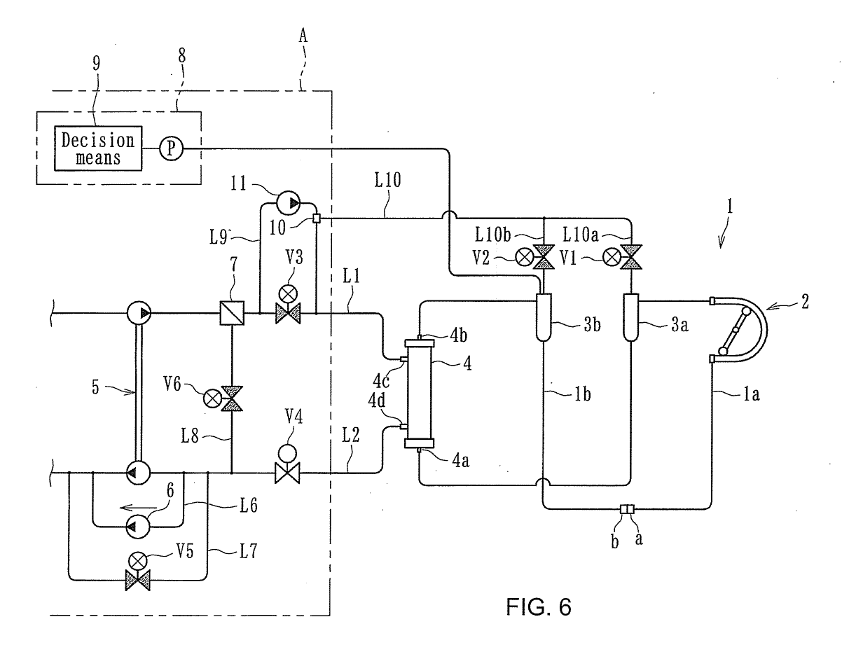

As shown in FIGS. 5 and 6, the blood purification apparatus of this embodiment includes a pressure varying mechanism to vary pressure in a closed circuit under a condition where the blood circuit 1 is formed as a closed circuit in a sealed condition connecting the tip end (connector) “a” of the arterial blood circuit 1a and the tip end (connector) “b” of the venous blood circuit 1b and under a condition where the blood circuit 1 is filled with priming solution. A liquid leakage detecting mechanism 8 is provided to detect liquid leakage in the blood circuit 1 in accordance with pressure variation generated by the pressure varying mechanism.

The pressure varying mechanism includes the substitution infusing pump (liquid supplying mechanism) 11 to increase liquid pressure in the closed circuit by applying a positive pressure to the closed circuit while introducing liquid (dialysate) into the closed circuit from the outside of the closed circuit. The ultrafiltration pump (liquid supplying mechanism) 6 decreases liquid pressure in the closed circuit by applying a negative pressure to the closed circuit while extracting liquid (priming solution (dialysate)) from the closed circuit to the outside of the closed circuit. The pressure varying mechanism is constructed so that it is possible to increase the liquid pressure in the closed circuit by applying a positive pressure to the closed circuit while introducing liquid (dialysate) into the closed circuit from the outside using the substitution infusing pump 11. Also, it is possible to decrease the liquid pressure in the closed circuit by applying a negative pressure to the closed circuit while extracting liquid (priming solution) from the closed circuit to the outside using the ultrafiltration pump 6. Thus, it is possible to perform the liquid leakage detection using the liquid leakage detecting mechanism 8 in the application of both the positive pressure and the negative pressure.

As described above, it is possible to apply the positive pressure to the closed circuit by driving the substitution infusing pump (liquid supplying mechanism) 11 while closing the electromagnetic valves V3, V4 and V5 and opening other electromagnetic valves as shown in FIG. 5. Thus, it is possible to introduce the dialysate in the dialysate introducing line L1 and the bypass line L9 to the arterial air trap chamber 3a and the venous air trap chamber 3b through the substitution infusing line L10 and branch lines L10a, L10b, respectively.

It is possible to apply the negative pressure to the closed circuit by forming the closed circuit in a sealed condition in the blood circuit 1 by closing the electromagnetic valves V1, V2 and accordingly the branch lines L10a, L10b. The ultrafiltration pump (liquid supplying mechanism) 6 is normally rotated while closing the electromagnetic valves V3, V5 and V6 and opening other electromagnetic valves as shown in FIG. 6. The priming is performed by filling the blood circuit 1 with priming solution (e.g. dialysate or physiological saline) under the connected condition of the tip end (connector “a”) of the arterial blood circuit 1a and the tip end (connector “b”) of the venous blood circuit 1b. Thus, the priming solution in the closed circuit to the dialysate discharging line L2 is extracted through the filtration membrane (hollow fiber membrane in the present embodiment) of the dialyzer 4. In this case, the substitution infusing pump 11 is stopped when applying the negative pressure to the closed circuit by normally driving the ultrafiltration pump (liquid supplying mechanism) 6. As described above, since the substitution infusing pump 11 is a displacement type pump, a flow path where the substitution infusing pump 11 is arranged is closed to prevent any flow of the dialysate.

Similar to the first embodiment, the liquid leakage detecting mechanism 8 includes the venous pressure sensor P as the liquid pressure detecting device and a decision mechanism 9. As previously described, the venous pressure sensor (liquid pressure detecting sensor) P is able to continuously (in real time) detect the venous pressure during the blood purification treatment (hemodialysis treatment). Additionally, it is able to continuously (in real time) detect the liquid pressure after the positive pressure has been applied to the closed circuit by the substitution infusing pump 11 and after the negative pressure has been applied to the closed circuit by the ultrafiltration pump 6.

The decision mechanism 9 is formed by e.g. a microcomputer etc. arranged within the main body “A” of the hemodialysis apparatus. It is electrically connected to the venous pressure sensor (liquid pressure detecting device) P to determine the existence of liquid leakage in the blood circuit 1 on the basis of liquid pressure detected by the venous pressure detecting device P. That is, the existence of liquid leakage is decided by the decision mechanism 9 in accordance with tendency after variation of the detected liquid pressure while continuously (in real time) detecting the tendency after variation of the liquid pressure caused by the pressure varying mechanism using the venous pressure sensor P. The method of determining the liquid leakage is the same as that of the first embodiment.

A method for inspecting liquid leakage of a dialysis apparatus of the second embodiment will be described.

First, the liquid pressure in the closed circuit is varied by the pressure varying mechanism under a condition where the blood circuit 1 is formed as a closed circuit in a sealed condition connecting the tip end of the arterial blood circuit is and the tip end of the venous blood circuit 1b and under a condition where the blood circuit 1 is filled with priming solution (pressure varying step). The liquid leakage in the blood circuit 1 is detected by the liquid leakage detecting mechanism in accordance with the variation of the liquid pressure in the pressure varying step (liquid leakage detecting step).

The liquid leakage detecting step includes a pressure detecting step for detecting liquid pressure in the closed circuit after a positive pressure or a negative pressure has been applied to the closed circuit and a decision step for deciding the existence of liquid leakage in the blood circuit 1 based on the liquid pressure detected in the pressure detecting step. In addition, in the pressure varying step, the liquid pressure in the closed circuit is increased by applying a positive pressure to the closed circuit by driving the substitution infusing pump 11 while introducing liquid into the closed circuit from the outside. Also, the pressure varying step procedure is decreased by applying a negative pressure to the closed circuit by normally rotating the ultrafiltration pump 6 while extracting liquid from the closed circuit to the outside. When the liquid leakage is detected in the liquid leakage detecting step, the fact is informed to an operator to urge him, confirming the liquid leakage.

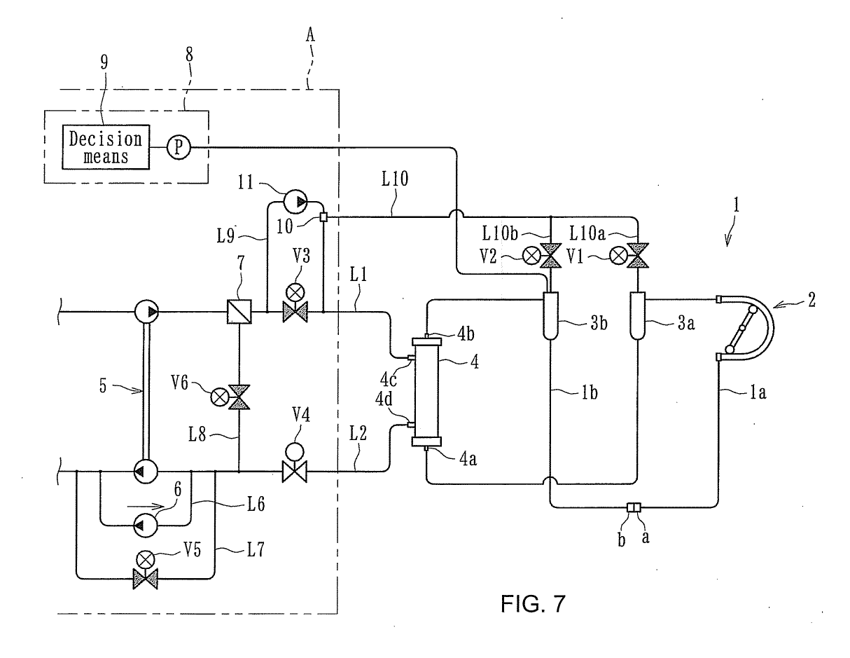

The ultrafiltration pump 6, functioning as the liquid supplying mechanism, is a pump able to rotate in both normal and reverse directions. Thus, either a positive pressure or a negative pressure can be applied to the closed circuit by selectively driving the ultrafiltration pump 6 in the normal direction or reverse direction. That is, as previously described, when the ultrafiltration pump 6 is normally rotated, it is possible to apply the negative pressure to the closed circuit. On the contrary, as shown in FIG. 7, when the ultrafiltration pump 6 is reversely rotated, the dialysate in the dialysate discharging line L2 is introduced into the flow path of the blood from the flow path of the dialysate of the dialyzer 4 through the filtration membrane (hollow fiber membrane in the present embodiment) (inverse filtration). Thus, the positive pressure can be applied to the closed circuit. According to such a construction, it is possible to easily and arbitrary apply the positive pressure and negative pressure to the closed circuit.

As described above, the pressure varying mechanism and the pressure varying step can vary the liquid pressure in the closed circuit by introducing liquid into the closed circuit from the substitution infusing line L10 or by extracting liquid from the closed circuit into the dialysate discharging line L2. The liquid leakage detecting mechanism and the liquid leakage detecting step can detect the liquid leakage of the substitution infusing line L10 or the dialysate discharging line L2. Liquid leakage can be detected in a positive pressure applying region of the substitution infusing line L10 or in a negative pressure applying region of the dialysate discharging line L2 in addition to the closed circuit. Accordingly, it is possible to detect liquid leakage of the substitution infusing line L10 or the dialysate discharging line L2 in addition to the closed circuit. Thus, the liquid leakage in a wide region can be achieved. Thus, it is possible to improve the reliability of the blood purification apparatus.

In the second embodiment, although it is described that the pressure varying step is performed under the condition where the priming solution is filled in the blood circuit 1, it may be performed under a condition where air, before the priming, is filled in the blood circuit 1. In this case, the liquid leakage is detected by the pressure varying mechanism varying atmosphere (pressure) in the closed circuit. The liquid leakage detecting mechanism detects the liquid leakage in accordance with the atmosphere variation.

A blood purification apparatus of a third embodiment will be described.

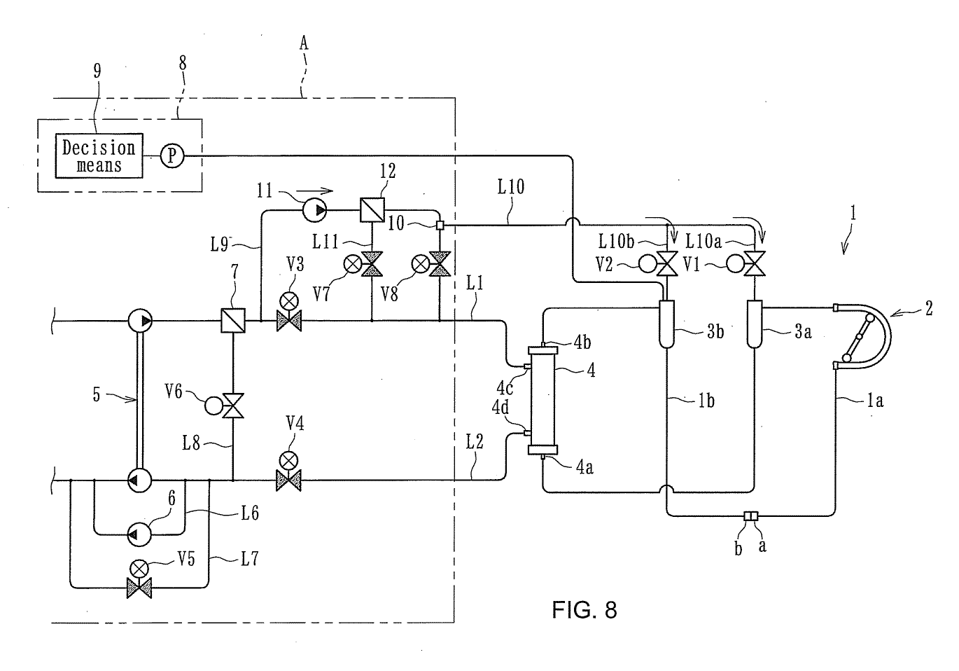

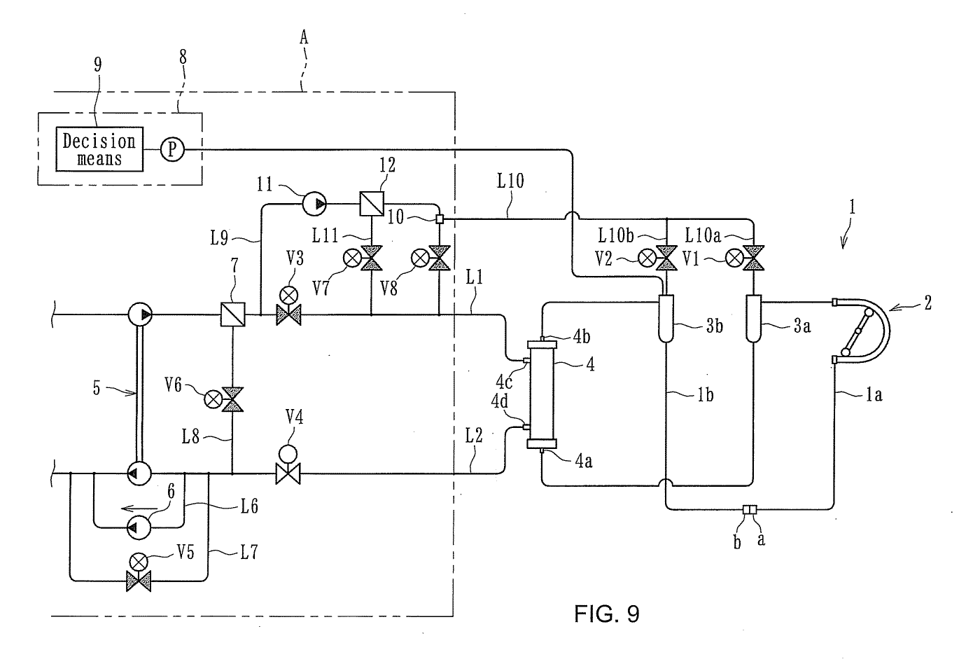

Similar to the first and second embodiments, the blood purification apparatus of this embodiment is adapted to be applied to a hemodialysis apparatus. It mainly includes, as shown in FIGS. 8 and 9, a blood circuit 1 including an arterial blood circuit is and a venous blood circuit 1b, a dialyzer 4 functioning as a blood purification instrument, a dialysate introducing line L1, a dialysate discharging line L2, a duplex pump 5, an ultrafiltration pump 6 functioning as liquid supplying mechanism forming a pressure varying mechanism, and a liquid leakage detecting mechanism 8. The same reference numerals will also be used to designate the same structural elements in the first and second embodiments and their detailed description will be omitted.

Similar to the second embodiment, a bypass line L9 bypassing the electromagnetic valve 3 is connected to the dialysate introducing line L1. A substitution infusing pump 11 and a substitution infusing port 10 are arranged on the way of the bypass line L9. The base end of a substitution infusing line L10 is connected to the substitution infusing port 10. The tip end of the substitution infusing line L10 is connected to the top portions (air layer) of the arterial air trap chamber 3a and the venous air trap chamber 3b, via a branch line L10a and a branch line L10b, respectively.

An electromagnetic valve V8 is arranged between the substitution infusing port 10 and the dialysate introducing line L1 on the bypass line L9. In addition, a flow path L11 is arranged between a filter 12 arranged on the bypass line L9 and the dialysate introducing line L1. Also, an electromagnetic valve V7 is arranged on the flow path L11.

The substitution infusing pump 11 is driven during the blood purification treatment. The dialysate is filtered by the filter 12 while flowing through the bypass line L9. The dialysate flowing through the dialysate introducing line L1 is introduced into the blood circuit 1 via the substitution infusing line L10 and the branch lines L10a, L10b. Thus, the substitution infusion is performed. Similar to the second embodiment, to perform the pre-dialysate during the blood purification treatment, the dialysate functioning as the substitution, is introduced into the arterial blood circuit 1a while opening the electromagnetic valve V1 and closing the electromagnetic valve V2. In order to perform the post-dialysate during the blood purification treatment, the dialysate, functioning as the substitution, is introduced into the venous blood circuit 1b while closing the electromagnetic valve V1 and opening the electromagnetic valve V2.

As shown in FIGS. 8 and 9, the blood purification apparatus of this embodiment includes a pressure varying mechanism to vary pressure in a closed circuit under a condition where the blood circuit 1 is formed as a closed circuit in a sealed condition connecting the tip end (connector) “a” of the arterial blood circuit 1a and the tip end (connector) “b” of the venous blood circuit 1b under a condition where the blood circuit 1 is filled with priming solution. A liquid leakage detecting mechanism 8 detects liquid leakage in the blood circuit 1 in accordance with pressure variation generated by the pressure varying mechanism.

The pressure varying mechanism includes the substitution infusing pump (liquid supplying mechanism) 11 to increase liquid pressure in the closed circuit by applying a positive pressure to the closed circuit while introducing liquid (dialysate) into the closed circuit from the outside of the closed circuit. The ultrafiltration pump (liquid supplying mechanism) 6 decreases liquid pressure in the closed circuit by applying a negative pressure to the closed circuit while extracting liquid (priming solution (dialysate)) from the closed circuit to the outside of the closed circuit. The pressure varying mechanism is constructed so that it is possible to increase the liquid pressure in the closed circuit by applying a positive pressure to the closed circuit while introducing liquid (dialysate) into the closed circuit from the outside using the substitution infusing pump 11. Also, it is possible to decrease the liquid pressure in the closed circuit by applying a negative pressure to the closed circuit while extracting liquid (priming solution) from the closed circuit to the outside using the ultrafiltration pump 6. Thus, it is possible to perform the liquid leakage detection using the liquid leakage detecting mechanism 8 in the application of both the positive pressure and the negative pressure.

As described above, it is possible to apply the positive pressure to the closed circuit by driving the substitution infusing pump (liquid supplying mechanism) 11 while closing the electromagnetic valves V3, V4, V5, V7 and V8 and opening other electromagnetic valves as shown in FIG. 8. Thus, the dialysate is introduced into the dialysate introducing line L1 and the bypass line L9 to the arterial air trap chamber 3a and the venous air trap chamber 3b through the substitution infusing line L10 and branch lines L10a, L10b, respectively.

It is possible to apply the negative pressure to the closed circuit by forming the closed circuit in a sealed condition in the blood circuit 1 by closing the electromagnetic valves V1, V2 and accordingly the branch lines L10a, L10b. The ultrafiltration pump (liquid supplying mechanism) 6 is normally rotated while closing the electromagnetic valves V3, V5, V6, V7 and V8 and opening other electromagnetic valves as shown in FIG. 9. Priming is performed by filling the blood circuit 1 with priming solution (e.g. dialysate or physiological saline) under the connected condition of the tip end (connector “a”) of the arterial blood circuit is and the tip end (connector “b”) of the venous blood circuit 1b. Thus, the priming solution is extracted in the closed circuit to the dialysate discharging line L2 through the filtration membrane (hollow fiber membrane in the present embodiment) of the dialyzer 4. In this case, the substitution infusing pump 11 is stopped when applying the negative pressure to the closed circuit by normally driving the ultrafiltration pump (liquid supplying mechanism) 6.

Similar to the second embodiment, the liquid leakage detecting mechanism 8 includes the venous pressure sensor P as a liquid pressure detecting device and a decision mechanism 9. As previously described, the venous pressure sensor (liquid pressure detecting device) P is able to continuously (in real time) detect the venous pressure during the blood purification treatment (hemodialysis treatment). Also, it is able to continuously (in real time) detect the liquid pressure after the positive pressure has been applied to the closed circuit by the substitution infusing pump 11 after the negative pressure has been applied to the closed circuit by the ultrafiltration pump 6.

The decision mechanism 9 is formed by e.g. a microcomputer etc. arranged within the main body “A” of the hemodialysis apparatus. It is electrically connected to the venous pressure sensor (liquid pressure detecting device) P to determine the existence of liquid leakage in the blood circuit 1 on the basis of liquid pressure detected by the venous pressure detecting device P. That is, the existence of liquid leakage is decided by the decision mechanism 9 in accordance with tendency after variation of the detected liquid pressure while continuously (in real time) detecting the tendency after variation of the liquid pressure caused by the pressure varying mechanism using the venous pressure sensor P. The method for deciding the liquid leakage is same as that of the first embodiment.

The method for inspecting liquid leakage of a dialysis apparatus of the third embodiment will be described.

First, the liquid pressure in the closed circuit is varied by the pressure varying mechanism under a condition where the blood circuit 1 is formed as a closed circuit in a sealed condition connecting the tip end of the arterial blood circuit is and the tip end of the venous blood circuit 1b under a condition where the blood circuit 1 is filled with priming solution (pressure varying step). Then, the liquid leakage in the blood circuit 1 is detected by the liquid leakage detecting sensor in accordance with the variation of the liquid pressure in the pressure varying step (liquid leakage detecting step).

The liquid leakage detecting step includes a pressure detecting step to detect liquid pressure in the closed circuit after a positive pressure or a negative pressure has been applied to the closed circuit. A decision step to determine the existence of liquid leakage in the blood circuit 1 based on the liquid pressure detected in the pressure detecting step. In addition, in the pressure varying step, the liquid pressure in the closed circuit is increased by applying a positive pressure to the closed circuit by driving the substitution infusing pump 11 while introducing liquid into the closed circuit from the outside. Also, the liquid pressure is decreased by applying a negative pressure to the closed circuit by normally rotating the ultrafiltration pump 6 while extracting liquid from the closed circuit to the outside. Similar to the first and second embodiments, it is preferable that the ultrafiltration pump 6, functioning as the liquid supplying mechanism, is a pump able to rotate in both normal and reverse directions. Thus, either a positive pressure or a negative pressure can be applied to the closed circuit by selectively driving the ultrafiltration pump 6 in the normal direction or reverse direction. When the liquid leakage is detected in the liquid leakage detecting step, the fact is informed to an operator to urge him, confirming the liquid leakage.

As described above, the pressure varying mechanism and the pressure varying step can vary the liquid pressure in the closed circuit by introducing liquid into the closed circuit from the substitution infusing line L10 or by extracting liquid from the closed circuit to the dialysate discharging line L2. The liquid leakage detecting mechanism and the liquid leakage detecting step can detect the liquid leakage of the substitution infusing line L10 or the dialysate discharging line L2. Liquid leakage is detected in a positive pressure applying region of the substitution infusing line L10, or in a negative pressure applying region of the dialysate discharging line L2 in addition to the closed circuit. Accordingly, since it is possible to detect liquid leakage of the substitution infusing line L10 or the dialysate discharging line L2 in addition to the closed circuit, the liquid leakage in a wide region can be achieved. Thus, it is possible to improve the reliability of the blood purification apparatus.

In the third embodiment, although it is described that the pressure varying step is performed under the condition where the priming solution is filled into the blood circuit 1, it may be performed under a condition where air, before the priming, is filled into the blood circuit 1. In this case, the liquid leakage is detected by the pressure varying mechanism varying atmosphere (pressure) in the closed circuit and the liquid leakage detecting mechanism detecting the liquid leakage in accordance with the atmosphere variation.

A blood purification apparatus of a fourth embodiment will be described.

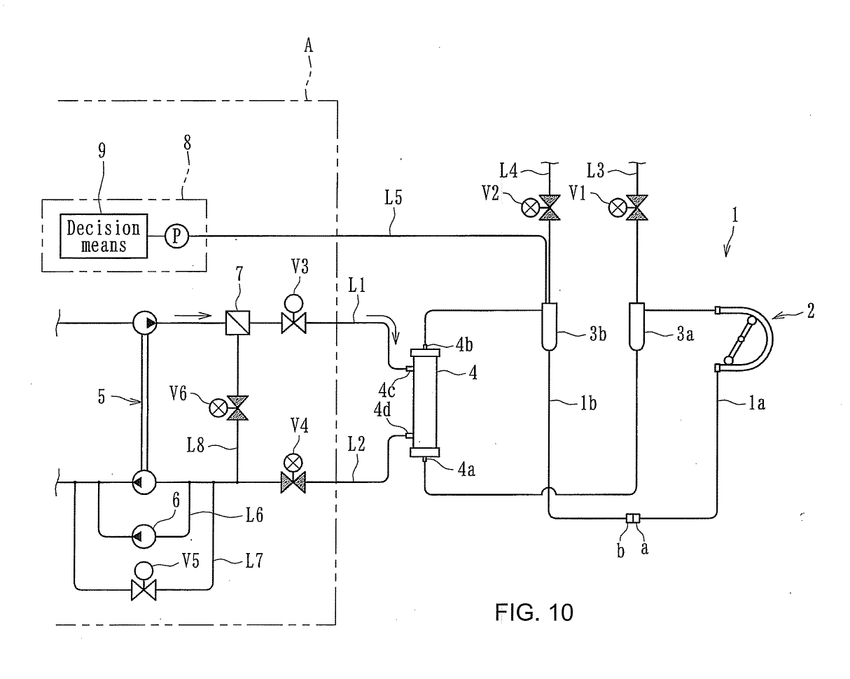

Similar to the first-third embodiments, the blood purification apparatus of this embodiment is adapted to be applied to a hemodialysis apparatus. Mainly it includes, as shown in FIGS. 10-13, a blood circuit 1 with an arterial blood circuit 1a and a venous blood circuit 1b, a dialyzer 4 functioning as a blood purification instrument, a dialysate introducing line L1, a dialysate discharging line L2, a duplex pump 5 functioning as liquid supplying mechanism forming a pressure varying mechanism, a liquid leakage detecting mechanism 8, and overflow lines L3, L4 as a discharging line. The same reference numerals will be used to designate the same structural elements in the first embodiment and their detailed description will be omitted.

The blood purification apparatus of this embodiment includes a pressure varying mechanism to vary pressure in a closed circuit under a condition where the blood circuit 1 is formed as a closed circuit in a sealed condition connecting the tip end (connector) “a” of the arterial blood circuit 1a and the tip end (connector) “b” of the venous blood circuit 1b. A liquid leakage detecting mechanism 8 is arranged in the closed circuit to detect liquid leakage in the blood circuit 1 in accordance with pressure variation generated by the pressure varying mechanism. The pressure varying mechanism includes the duplex pump (liquid supplying mechanism) 5 to increase liquid pressure in the closed circuit by applying a positive pressure to the closed circuit while introducing liquid (dialysate) into the closed circuit from the outside of the closed circuit.

As described above, the tip end (connector) “a” of the arterial blood circuit 1a and the tip end (connector) “b” of the venous blood circuit 1b are connected together. Thus, a closed and sealed circuit is formed in the blood circuit I. In this embodiment, the closed circuit is filled with air before priming while with closing the electromagnetic valves V1, V2 to close the overflow lines L3, L4 as shown in FIG. 10. The duplex pump (liquid supplying mechanism) 5, drives dialysate in the dialysate introducing line L1 (portion between the duplex pump 5 and the dialyzer 4) and the dialysate discharging line L2 (portion between the dialyzer 4 and the electromagnetic valve V4) into a blood flow path from a dialysate flow path of the dialyzer 4 through the filtration membrane (hollow fiber membrane in the present embodiment) of the dialyzer 4 (inverse filtration). Thus, it is possible to apply the positive pressure to the closed circuit.

The overflow lines L3, L4, as discharging lines, include flow paths able to discharge liquid or gas outside of the closed circuit. Also, they are able to be opened and closed by the electromagnetic valves V1, V2. The overflow lines L3, L4 are closed by closing the electromagnetic valves V1, V2. Thus, the closed circuit is formed. On the other hand, when opening either one of the electromagnetic valves V1, V2, the closed condition of the overflow lines L3, L4 is released and liquid or gas is discharged to the outside.

The method for inspecting liquid leakage of a dialysis apparatus of the fourth embodiment will be described.

First, the liquid pressure in the closed circuit is varied by the pressure varying mechanism under a condition where the blood circuit 1 is formed as a closed circuit in a sealed condition (pre-priming condition) connecting the tip end of the arterial blood circuit 1a and the tip end of the venous blood circuit 1b (pressure varying step). Then, the liquid leakage in the blood circuit 1 is detected by the liquid leakage detecting mechanism in accordance with the variation of the liquid pressure in the pressure varying step (liquid leakage detecting step).

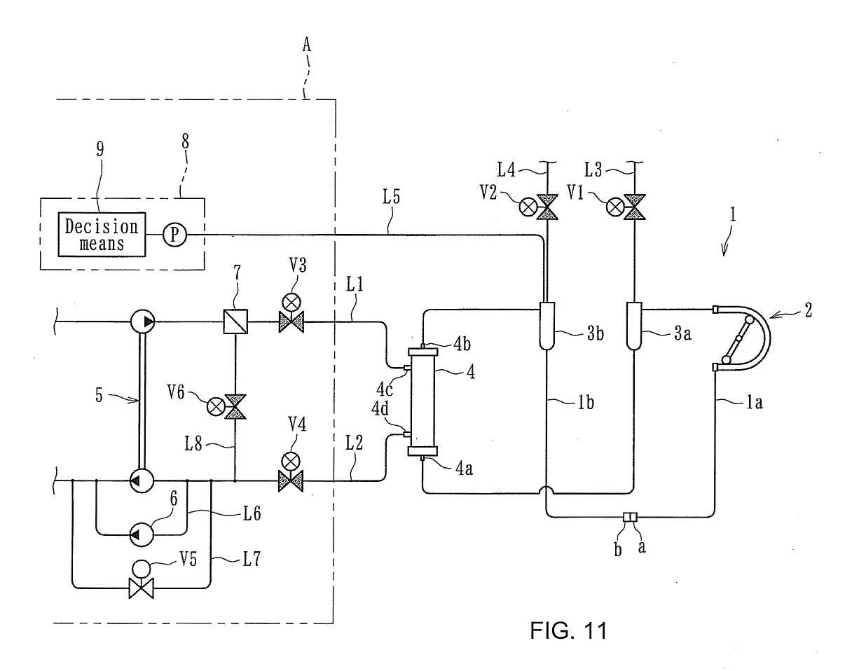

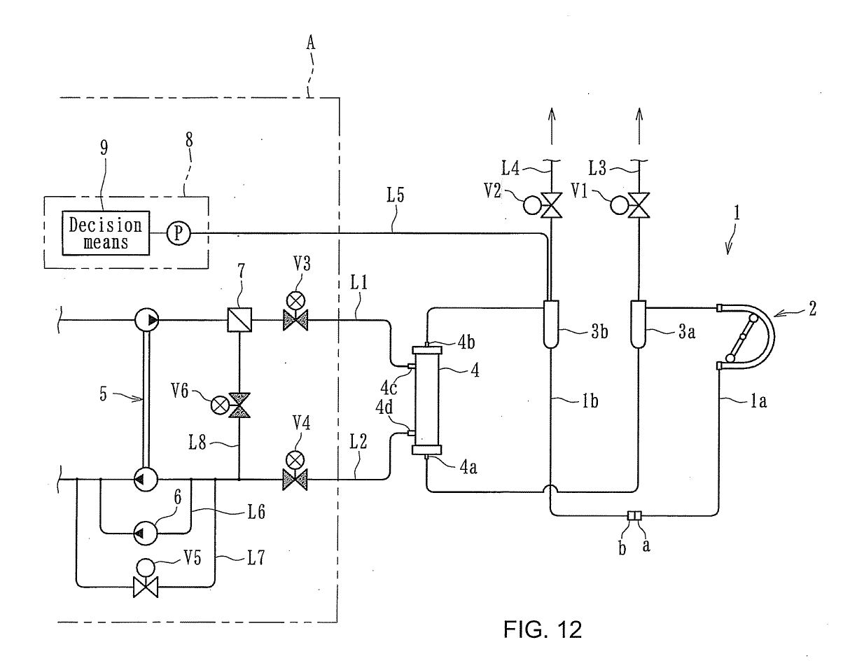

The pressure varying step of this embodiment is constructed so that the liquid leakage detecting step (see FIG. 11) to detect the liquid leakage in the blood circuit by the liquid leakage detecting mechanism during the pressurizing step, is performed by alternately performing the pressurizing step (see FIG. 10). Here, the positive pressure is applied to the closed circuit by introducing liquid (dialysate) into the closed circuit from the outside by the duplex pump (liquid supplying mechanism) 5 while closing the overflow lines L3, L4. The priming step (see FIG. 12) where liquid or gas in the closed circuit is discharged from the overflow lines L3, L4 occurs while opening the overflow lines L3, L4 and introducing liquid into the closed circuit from the outside by the duplex pump 5.

More particularly, as shown in FIG. 10, the pressurizing step is a step intended to increase the pressure in the closed circuit by applying the positive pressure to the closed circuit while closing V1, V2, V4 and V6 and opening other electromagnetic valves with inverse filtrating dialysate entering into the dialysate introducing line L1 by the duplex pump 5. In the pressurizing step, it is possible to apply the positive pressure to the closed circuit by introducing dialysate into the closed circuit by reversely rotating the ultrafiltration pump 6 in place of the duplex pump 5. Here, the electromagnetic valve V4 is opened and the electromagnetic valve V3 is closed.

The decision step is a step performed during the pressurizing step and constructed as shown in FIG. 11 so that the existence of liquid leakage in the blood circuit 1 is determined by detecting the pressure in the closed circuit by the venous pressure sensor P (pressure detecting device) while closing the electromagnetic valve V3. The decision mechanism 9 is based on the detected value. In the decision step, although the duplex pump 5 is kept in a stopped condition, it is possible to keep the operation of the duplex pump 5 with the opening the electromagnetic valve V6.

According to this embodiment, a portion between the dialyzer 4 and the electromagnetic valve V3 on the dialysate introducing line L1 and a portion between the dialyzer 4 and the electromagnetic valve V4 on the dialysate discharging line L2 are included in the closed circuit in addition to the blood circuit 1. Thus, it is possible to determine the existence of liquid leakage of the dialysate introducing line L1 and the dialysate discharging line L2.

The priming step is a step performed after the pressurizing step and constructed as shown in FIG. 12. The pressure in the closed circuit is released to e.g. the normal pressure by discharging outside gas (air) or liquid (dialysate) in the closed circuit from the overflow lines L3, L4 while opening the electromagnetic valves V1, V2. Thus, it is possible to discharge air (including air bubbles in the dialysate) in the closed circuit from the overflow lines L3, L4 during the priming step. Similar to the liquid leakage detecting step, also in the priming step, although the duplex pump 5 is kept in a stopped condition, it is possible to keep the operation of the duplex pump 5 with the opening of the electromagnetic valve V6.

According to this embodiment, the dialysate, functioning as priming solution, fills the closed circuit with alternately repeating a number of predetermined times the pressurizing step and the priming step. However, since it is difficult to fill, with dialysate, a portion between the arterial air trap chamber 3a and the venous air trap chamber 3b, a circulating step is performed as follows.

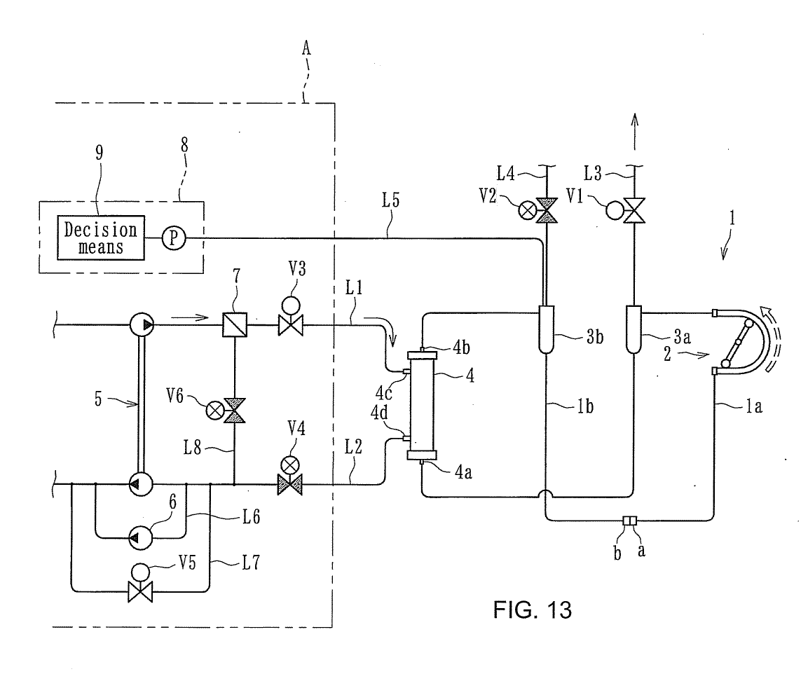

As shown in FIG. 13, the circulating step is a step to discharge air (including air bubbles in the dialysate) in the closed circuit from the overflow line L3 by driving the duplex pump 5. The blood pump 2 circulates dialysate in the closed circuit with the opening of the electromagnetic valve V1 and the closing of the electromagnetic valve V2 as well as with the closing of the electromagnetic valves V4, V6 and opening other electromagnetic valves. Such a circulating step fills all flowing paths forming the closed circuit with dialysate as the priming solution.

As described above, in this embodiment, the dialysate as the priming solution, fills the closed circuit by alternately repeating a number of predetermined times the pressurizing step and the priming step. The pressurizing step is usually performed several times. The liquid leakage detecting step may be performed at all times during performing of the pressurizing step or may be performed at only a predetermined time (e.g. at a time of final pressurizing time).

In this case, it is possible to achieve a finer liquid leakage detection if the liquid leakage detecting step is performed at all times during a plurality of pressurizing steps. Also, it is possible to achieve more exact liquid leakage detection if the liquid leakage detection step is performed only when the priming solution (dialysate) fills the closed circuit. In addition, although it is described that the liquid leakage detecting step of this embodiment is performed while keeping the electromagnetic valve V3 closed (see FIG. 11), it may be possible to perform the liquid leakage detection while keeping the opened condition of the electromagnetic valve V3 in the pressurizing step.

According to the first-fourth embodiments, the liquid leakage in the blood circuit 1 is detected by varying pressure in the closed circuit connecting the tip end of the arterial blood circuit and the tip end of the venous blood circuit. Thus, it is possible to perform a sufficient liquid leakage inspection over a whole region (whole region including the tip end side of the arterial blood circuit is and the tip end side of the venous blood circuit 1b) of the flow path of the blood circuit 1. More particularly, according to the first-third embodiments, the liquid leakage in the blood circuit 1 is detected by varying the liquid pressure in the closed circuit under a condition where the blood circuit 1 is filled with priming liquid. Thus, it is possible to perform a sufficient liquid leakage inspection over a whole region of the blood circuit 1.

According to the fourth embodiment, the blood purification apparatus further includes overflow lines L3, L4 as discharging lines to discharge liquid or gas in the closed circuit to the outside. The liquid leakage in the blood circuit 1 is detected during a pressurizing step by alternately performing the pressurizing step and a priming step. In the pressurizing step, the positive pressure is applied to the closed circuit by introducing liquid into the closed circuit from the outside. The duplex pump 5, as a liquid supplying mechanism, while keeping a closed condition of the overflow lines L3, L4, introduces the priming step liquid into the closed circuit from the outside. The duplex pump 5, while keeping an opened condition of the overflow lines L3, L4, enables liquid or gas in the closed circuit to be discharged from the overflow lines L3, L4. Thus, it is possible to perform the detection of the liquid leakage during a process where the priming (discharge of air bubbles and filling of priming liquid) is performed in the blood circuit 1.

Furthermore, the liquid leakage detection is performed by detecting pressure in the closed circuit after a positive pressure has been applied to the closed circuit. The existence of liquid leakage is determined in the blood circuit 1 on the basis of the detected pressure. Thus, it is possible to detect the pressure when the positive pressure is applied to the closed circuit by substituting a venous pressure sensor P, usually connected to the venous air trap chamber 3b connected to the venous blood circuit 1b, for a positive pressure detecting sensor. Thus, this reduces the manufacturing cost of the blood purification apparatus.

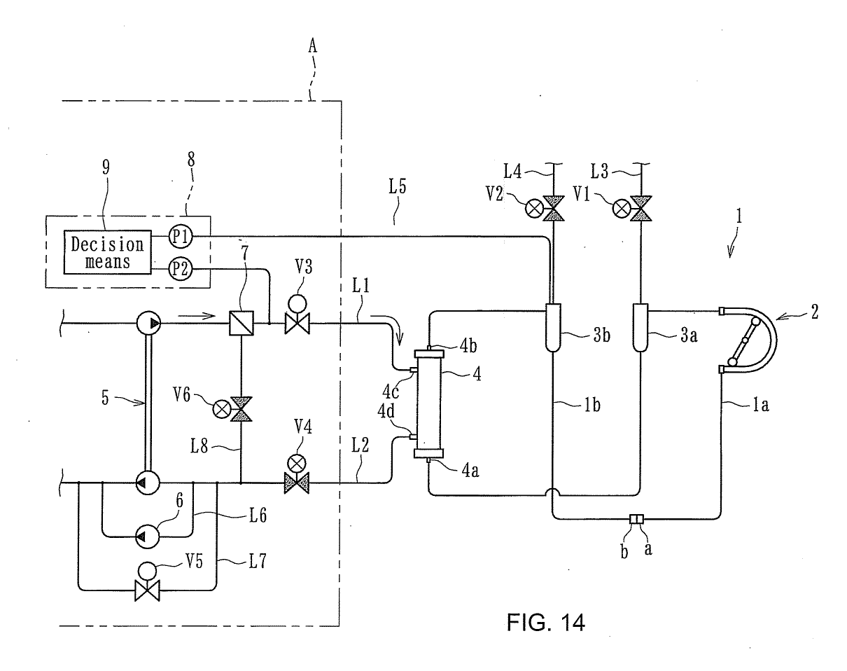

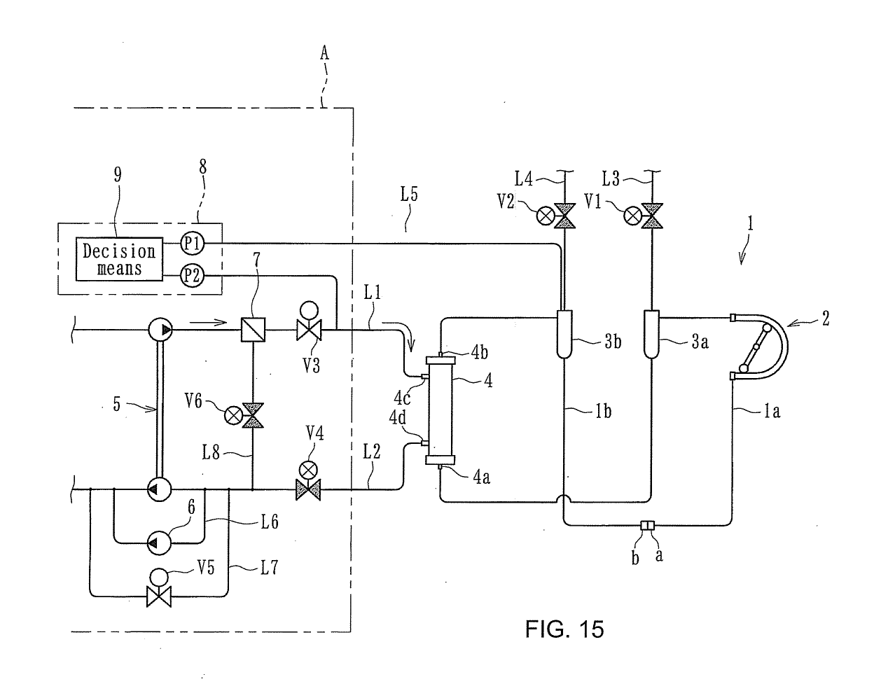

However, the existence of liquid leakage can be detected by modifications shown in FIGS. 14 and 15. In the modification of FIG. 14, a pressure detecting mechanism (venous pressure sensor) P1 is connected to the venous air trap chamber 3b in the closed circuit. Another pressure detecting mechanism P2 is connected to a portion upstream of the electromagnetic valve V3 (between the electromagnetic valve V3 and the filter 7) on the dialysate introducing line L1. The existence of liquid leakage can be determined by the decision mechanism 9 by comparing pressures detected by the pressure detecting mechanism P1, P2. In the modification of FIG. 15, a pressure detecting mechanism (venous pressure sensor) P1 is connected to the venous air trap chamber 3b in the closed circuit. Another pressure detecting mechanism P2 is connected to a portion downstream of the electromagnetic valve V3 (between the electromagnetic valve V3 and the dialyzer 4) on the dialysate introducing line L1. The existence of liquid leakage can be determined by the decision mechanism 9 by comparing pressures detected by the pressure detecting mechanism P1, P2.

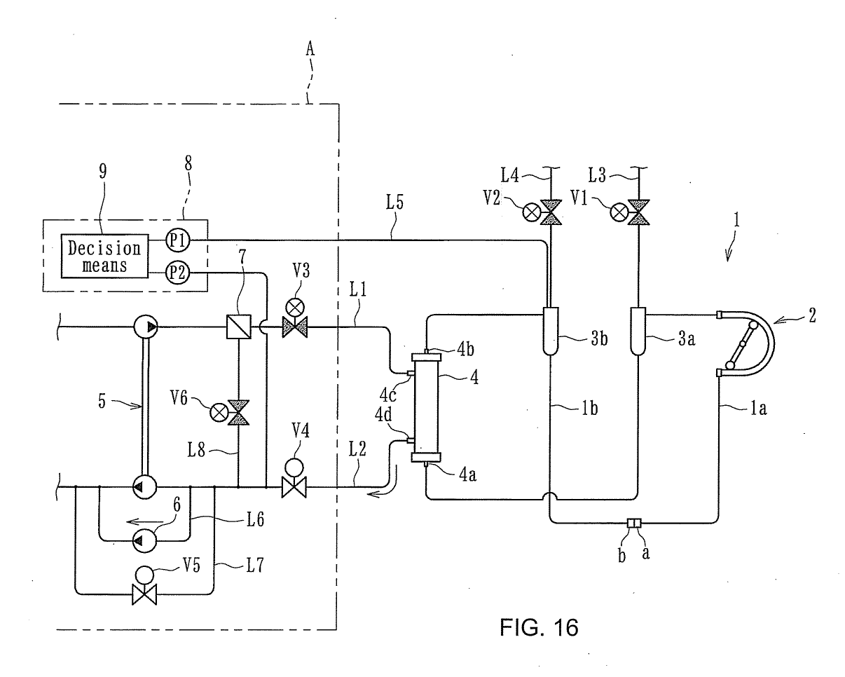

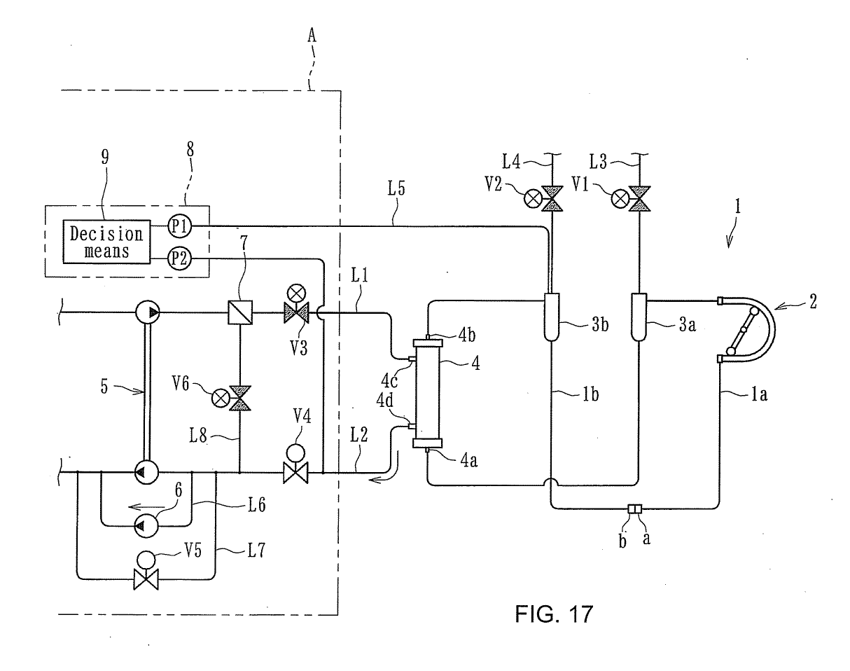

In addition, the existence of liquid leakage can be detected by modifications shown in FIGS. 16 and 17. In the modification of FIG. 16, a pressure detecting mechanism (venous pressure sensor) P1 is connected to the venous air trap chamber 3b in the closed circuit. Another pressure detecting mechanism P2 is connected to a portion downstream of the electromagnetic valve V4 (between the electromagnetic valve V4 and the bypass line L6) on the dialysate discharging line L2. The existence of liquid leakage can be determined by the decision mechanism 9 by comparing pressures detected by the pressure detecting mechanism P1, P2. In the modification of FIG. 17, a pressure detecting mechanism (venous pressure sensor) P1 is connected to the venous air trap chamber 3b in the closed circuit. Another pressure detecting mechanism P2 is connected to a portion upstream of the electromagnetic valve V4 (between the electromagnetic valve V4 and the dialyzer 4) on the dialysate introducing line L1. The existence of liquid leakage can be determined by the decision mechanism 9 by comparing pressures detected by the pressure detecting mechanism P1, P2.

As described above, the pressure detecting mechanism P1, P2 are arranged, respectively, on the closed circuit and on the dialysate introducing line L1 or the dialysate discharging line L2. The decision mechanism 9 decides the existence of liquid leakage in the blood circuit 1 by comparing pressures detected by the pressure detecting mechanism P1, P2. Thus, it is possible to further improve the accuracy of the decision of the liquid leakage.

Thus, it is possible to decide malfunction of either one of the pressure detecting mechanism, clog etc. in the flow paths of the blood circuit 1, dialysate introducing line L1 or the dialysate discharging line L2. Thus, this improves the deciding accuracy of the liquid leakage whether monitoring a pressure detected by the pressure detecting mechanism P1 corresponding to a pressure detected by the pressure detecting mechanism P2. Thus, it is possible to improve the reliability of the blood purification apparatus.

Furthermore, the pressure variation applied to the closed circuit includes both the pressure increase obtained by applying the positive pressure to the closed circuit while introducing liquid (dialysate) to the closed circuit from the outside and the pressure decrease obtained by applying the negative pressure to the closed circuit while extracting liquid (dialysate) from the closed circuit to the outside. Accordingly, the detection of the liquid leakage can be performed when both the positive pressure and the negative pressure are applied. Thus, more suitable and sufficient liquid leakage inspection can be achieved. However, in this case it is preferable to apply the positive pressure to the closed circuit after application of the negative pressure since this prevents suction of air from the tip ends of the arterial blood circuit 1a and the venous blood circuit when the connection is released to smoothly perform the blood purification treatment.

In addition, according to the first˜fourth embodiment, the introduction and discharge of liquid to or from the closed circuit is performed by the duplex pump (dialysate pump) 5 introducing dialysate into the dialyzer (blood purification instrument) 4. The ultrafiltration pump 6 performs ultrafiltration against blood extracorporeally circulating through the blood circuit 1. The substitution infusing pump 11 can also be used. Thus, it is possible to substitute pumps used in the blood purification treatment for these pumps. This reduces the manufacturing cost of the blood purification apparatus. The liquid supplying mechanism is not limited to pumps and other mechanism (including those not driven during the blood purification treatment) can be used if they can apply the positive pressure or the negative pressure by introducing or extracting liquid (dialysate) to or from the closed circuit.

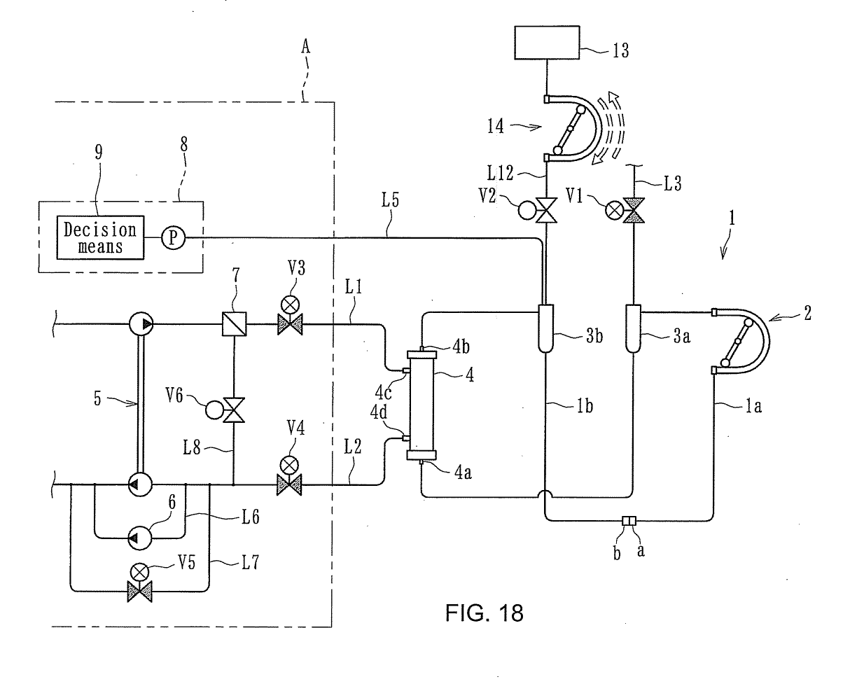

Although preferable embodiments and modifications have been described above, the present disclosure is not limited to these embodiments and modifications. For example, as shown in FIG. 18, the present disclosure can be applied to a construction where a physiological saline line L12 extends from an upper portion (air layer) of the venous air trap chamber 3b to a physiological saline bag 13 containing a predetermined amount of physiological saline. The electromagnetic valve V2 and a solution infusing pump 14, as a liquid supplying mechanism are arranged on the physiological saline line L12. Thus, the physiological saline in the physiological saline bag 13 can be introduced into the venous blood circuit 1b through the venous air trap chamber 3b.

Thus, as shown in FIG. 18, it is possible to apply the positive pressure to the closed circuit by closing the electromagnetic valves V1, V3, V4 and V5 and opening other electromagnetic valves. The substitution infusing pump (liquid supplying mechanism) 14 introduces liquid (physiological saline) in the physiological saline bag 13 into the venous air trap chamber 3b via the physiological saline line L12. This occur after performing the priming by filling the blood circuit 1 with the priming solution (e.g. physiological saline in the physiological saline bag 13) under a condition of formation of the closed circuit in a sealed condition connecting the tip end (connector “a”) of the arterial blood circuit 1a and the tip end (connector “b”) of the venous blood circuit 1b.

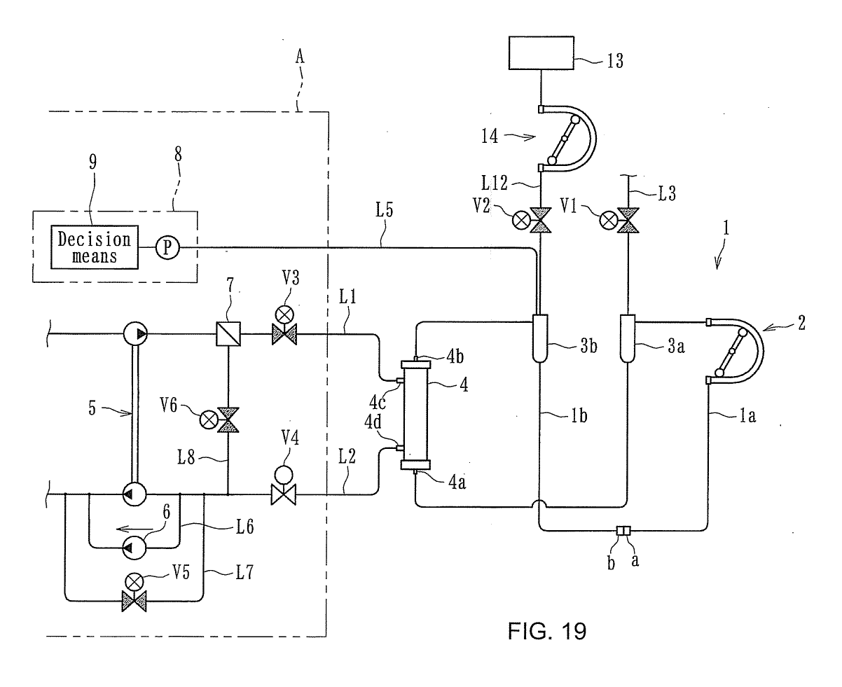

On the other hand, the negative pressure can be applied to the closed circuit as shown in FIG. 19 by forming the sealed closed circuit in the blood circuit 1 while closing the electromagnetic valves V1, V2 to close the overflow line L3 and the physiological saline line L12. The ultrafiltration pump (liquid supplying mechanism) 6 with the closing of the electromagnetic valves V3, V5 and V6 and opening of other electromagnetic valves introduces the priming solution (dialysate or physiological saline) into the closed circuit into the dialysate discharging line L2 through the filtration membrane (hollow fiber membrane in this embodiment) of the dialyzer 4.

It is possible to apply the negative pressure to the closed circuit by reversely rotating the substitution infusing pump (liquid supplying mechanism) 14 to supply the liquid (priming solution) to the physiological saline line L12. As shown in FIG. 19, it is possible to apply the positive pressure to the closed circuit by reversely rotating the ultrafiltration pump 6 to introduce the liquid (dialysate) to the closed circuit.

Furthermore, according to the present disclosure, although it is described that both the positive pressure and negative pressure are applied to the closed circuit to perform the liquid leakage inspection, it may be possible to perform either one of the positive pressure application or the negative pressure application. The liquid pressure variation to the closed circuit is performed by applying the positive pressure to the closed circuit while introducing liquid into the closed circuit from the outside. Thus, it is possible to prevent air from being sucked into the blood circuit 1 when the liquid leakage would be caused in the negative pressure inspection. Thus, it is possible to smoothly and properly perform the blood purification treatment.

In addition, the liquid pressure variation relative to the closed circuit is performed by applying the negative pressure to reduce liquid pressure while extracting liquid to the outside of the closed circuit. Thus, it is possible to perform the liquid leakage inspection due to the negative pressure of a portion where the negative pressure is applied (e.g. a portion nearer to the tip end “a” than the blood pump 2 in the arterial blood circuit) in the blood purification treatment. Thus, it is possible to perform the liquid leakage inspection based on actions applied during the blood purification treatment.

Furthermore, the liquid pressure variation relative to the closed circuit is performed by applying the negative pressure to reduce liquid pressure while extracting liquid to the outside of the closed circuit. The liquid leakage detecting mechanism may be formed of an air bubble detecting mechanism able to detect air bubbles generated in the case of the liquid leakage in the blood circuit 1. A decision mechanism determines the existence of the liquid leakage in the blood circuit 1 based on detecting the bubbles with the air bubble detecting mechanism. In this case, it is possible to use, as a liquid pressure detector, for detecting the negative pressure to the closed circuit, an air bubble detector etc. usually connected to a portion nearer the tip end “b” than the air trap chamber 3b in the venous blood circuit 1b. This reduces the manufacturing cost of the blood purification apparatus. The liquid leakage can also be detected by visual observation based on air bubbles since the air bubbles are contained in liquid.

Although the present disclosure has been described with reference to several preferable embodiments, it is sufficient if it can detect the liquid leakage at least in the blood circuit. The present disclosure can be applied to other liquid circuit (liquid flow path) than the blood circuit. In addition, the present disclosure can be applied to other blood purification apparatus other than the hemodialysis apparatus. Furthermore, although it is described that the present disclosure is applied to the observation apparatus for dialysis (not having dialysate preparing function), it is apparent that the present disclosure can be applied to the private dialysis apparatus (having dialysate preparing function).

The present disclosure can be applied to any other applications having additional functions if they are blood purification apparatus and methods for inspecting liquid leakage adapted to detect liquid leakage in the blood circuit by varying pressure in the closed circuit formed by connecting the tip ends of an arterial blood circuit and a venous blood circuit.

The present disclosure has been described with reference to the preferred embodiments. Obviously, modifications and alternations will occur to those of ordinary skill in the art upon reading and understanding the preceding detailed description. It is intended that the present disclosure be construed to include all such alternations and modifications insofar as they come within the scope of the appended claims or their equivalents.

What is claimed is:

1. A blood purification apparatus comprising:
a blood circuit including an arterial blood circuit and a venous blood circuit, a blood pump arranged on the blood circuit for extracorporeally circulating blood of a patient;
a blood purification instrument for purifying the blood of a patient extracorporeally circulated through the blood circuit, the blood purification instrument connected with a base end of the arterial blood circuit and a base end of the venous blood circuit of the blood circuit;
a dialysate introducing line for introducing dialysate into the blood purifying instrument;
a dialysate discharging line for discharging the dialysate from the blood purifying instrument;
a pressure varying mechanism for varying pressure in a closed circuit under a condition where the blood circuit is formed as the closed circuit in a sealed condition connecting the tip end of the arterial blood circuit and the tip end of the venous blood circuit; and
a liquid leakage detecting mechanism arranged in the closed circuit for detecting liquid leakage in the blood circuit in accordance with pressure variation generated by the pressure varying mechanism.

2. The blood purification apparatus of claim 1, wherein the pressure varying mechanism varies liquid pressure in the closed circuit under a condition where the blood circuit is filled with priming liquid.

3. The blood purification apparatus of claim 1, wherein the pressure varying mechanism comprises a liquid supplying mechanism for increasing pressure in the closed circuit by applying a positive pressure to the closed circuit while introducing liquid into the closed circuit from the outside.

4. The blood purification apparatus of claim 3, wherein the blood purification apparatus further comprises a discharging line for discharging liquid or gas in the closed circuit to the outside; and
the liquid leakage in the blood circuit is detected by the liquid leakage detecting mechanism during a pressurizing step by alternately performing the pressurizing step and a priming step, in the pressurizing step the positive pressure is applied to the closed circuit by introducing liquid into the closed circuit from the outside by the liquid supplying mechanism while keeping the discharging line in a closed condition, and in the priming step, liquid is introduced into the closed circuit from the outside by the liquid supplying mechanism while keeping the discharging line in an opened condition and liquid or gas in the closed circuit is discharged from the discharging line.

5. The blood purification apparatus of claim 3, wherein the liquid leakage detecting mechanism comprises:
a pressure detecting device for detecting pressure in the closed circuit after a positive pressure has been applied to the closed circuit; and
a decision mechanism for determining the existence of liquid leakage in the blood circuit based on the pressure detected by the pressure detecting device.

6. The blood purification apparatus of claim 1, wherein the pressure varying mechanism comprises a liquid supplying mechanism for decreasing pressure in the closed circuit by applying a negative pressure to the closed circuit while extracting liquid from the closed circuit to the outside.

7. The blood purification apparatus of claim 6, wherein the liquid leakage detecting mechanism comprises:
a pressure detecting device for detecting pressure in the closed circuit after a negative pressure has been applied to the closed circuit; and
a decision mechanism for determining the existence of liquid leakage in the blood circuit based on the detected pressure by the pressure detecting device.

8. The blood purification apparatus of claim 5, wherein the pressure detecting device comprises one sensor arranged in the closed circuit and another sensor arranged in the dialysate introducing line or the dialysate discharging line, and wherein the decision mechanism determines the existence of liquid leakage in the blood circuit by comparing a pressure detected by the pressure detecting sensor arranged in the closed circuit with a pressure detected by the pressure detecting sensor arranged in the dialysate introducing line or the dialysate discharging line.

9. The blood purification apparatus of claim 6, wherein the liquid leakage detecting mechanism comprises:
an air bubble detecting device for detecting bubbles that would be generated during liquid leakage in the blood circuit when the negative pressure is applied to the closed circuit; and
a decision mechanism for determining the existence of liquid leakage based on the basis of a fact whether the air bubbles are detected.

10. The blood purification apparatus of claim 3, wherein the liquid supplying mechanism comprises a dialysate pump for introducing the dialysate into the blood purifying instrument, an ultrafiltration pump for performing ultrafiltration against blood circulating extracorporeally through the blood circuit, or a substitution infusing pump for introducing a substitution to the blood circuit.

11. The blood purification apparatus of claim 10, wherein the liquid supplying mechanism is a pump able to perform a normal rotation and a reverse rotation, and either the application of positive pressure or negative pressure to the closed circuit can be achieved by selectively performing the normal rotation or the reverse rotation.

12. The blood purification apparatus of claim 1, wherein the pressure varying mechanism is able to increase pressure in the closed circuit by applying a positive pressure to the closed circuit while introducing liquid into the closed circuit from the outside and is also able to decrease pressure in the closed circuit by applying a negative pressure to the closed circuit while extracting liquid from the closed circuit to the outside, and the liquid leakage detecting mechanism is able to detect liquid leakage in both cases of application of the positive pressure and negative pressure.

13. The blood purification apparatus of claim 12, wherein the positive pressure is applied to the closed circuit after the negative pressure has been applied to the closed circuit.

14. The blood purification apparatus of claim 1, wherein the pressure varying mechanism varies pressure in the closed circuit by introducing liquid into the closed circuit through the dialysate introducing line or by discharging liquid from the closed circuit to the dialysate discharging line, and the liquid leakage detecting mechanism is able to detect liquid leakage in the dialysate introducing line or the dialysate discharging line in addition to detecting liquid leakage in the closed circuit.

15. A method for inspecting liquid leakage of a blood purification apparatus including a blood circuit with an arterial blood circuit and a venous blood circuit, a blood pump for extracorporeally circulating blood of a patient, a blood purification instrument for purifying the blood of a patient extracorporeally circulated through the blood circuit, the blood purification instrument is connected with a base end of the arterial blood circuit and a base end of the venous blood circuit of the blood circuit, a dialysate introducing line introduces dialysate into the blood purifying instrument, and a dialysate discharging line discharges the dialysate from the blood purifying instrument;
a pressure varying step varying pressure in a closed circuit under a condition where the blood circuit is formed as a closed circuit in a sealed condition connecting the tip end of the arterial blood circuit and the tip end of the venous blood circuit; and
a liquid leakage detecting step detecting liquid leakage in the blood circuit in accordance with a pressure variation during varying pressure varying.

16. The method for inspecting liquid leakage of a blood purification apparatus of claim 15, wherein during the varying pressure step in the closed circuit, the liquid pressure in the closed circuit is varied under a condition where the blood circuit is filled with priming liquid.

17. The method for inspecting liquid leakage of a blood purification apparatus of claim 16, wherein in the pressure varying step, the pressure in the closed circuit is increased by applying a positive pressure to the closed circuit while introducing liquid into the closed circuit from the outside.

18. The method for inspecting liquid leakage of a blood purification apparatus of claim 17:
wherein the blood purification apparatus further comprises a discharging line for discharging liquid or gas in the closed circuit to the outside; and
the liquid leakage detecting step is performed during a pressurizing step by alternately performing the pressurizing step and a priming step, in the pressurizing step the positive pressure is applied to the closed circuit by introducing liquid into the closed circuit from the outside by the liquid supplying mechanism while keeping the discharging line in a closed condition, and the priming step liquid is introduced into the closed circuit from the outside by the liquid supplying mechanism while keeping the discharging line in an opened condition and liquid or gas in the closed circuit is discharged from the discharging line.

19. The method for inspecting liquid leakage of a blood purification apparatus of claim 17, wherein the liquid leakage detecting step comprises:
a pressure detecting step for detecting pressure in the closed circuit after a positive pressure has been applied to the closed circuit; and
a decision step for determining the existence of liquid leakage in the blood circuit based on the basis of the pressure detected in the pressure detecting step.

20. The method for inspecting liquid leakage of a blood purification apparatus of claim 15, wherein in the pressure varying step, the pressure in the closed circuit is decreased by applying a negative pressure to the closed circuit while extracting liquid from the closed circuit to the outside.

21. The method for inspecting liquid leakage of a blood purification apparatus of claim 20, wherein the liquid leakage detecting step comprises:
a pressure detecting step for detecting pressure in the closed circuit after a negative pressure has been applied to the closed circuit; and
a decision step for determining the existence of liquid leakage in the blood circuit based on pressure detected in the pressure detecting step.

22. The method for inspecting liquid leakage of a blood purification apparatus of claim 19, wherein in the pressure detecting step, pressures in the closed circuit and in the dialysate introducing line or the dialysate discharging line are detected, respectively, and the liquid leakage in the blood circuit is decided by comparing a pressure detected in the closed circuit with a pressure detected in the dialysate introducing line or the dialysate discharging line.

23. The method for inspecting liquid leakage of a blood purification apparatus of claim 20, wherein the liquid leakage detecting step comprises:
an air bubble detecting step for detecting bubbles that are generated in case of liquid leakage in the blood circuit when the negative pressure is applied to the closed circuit; and
a decision step for deciding the existence of liquid leakage based on whether the air bubbles are detected.

24. The method for inspecting liquid leakage of a blood purification apparatus of claim 17, wherein the introduction or discharge of liquid into or from the closed circuit in the pressure varying step is performed by a dialysate pump for introducing the dialysate to the blood purifying instrument, an ultrafiltration pump for performing ultrafiltration against blood circulating extracorporeally through the blood circuit, or a substitution infusing pump for introducing a substitution into the blood circuit.

25. The method for inspecting liquid leakage of a blood purification apparatus of claim 24, wherein the introduction or discharge of liquid into or from the closed circuit in the pressure varying step is achieved by a pump being able to perform a normal rotation and a reverse rotation, and either the application of positive pressure or negative pressure to the closed circuit can be achieved by selectively performing the normal rotation or the reverse rotation.

26. The method for inspecting liquid leakage of a blood purification apparatus of claim 15, wherein in the pressure varying step, the pressure in the closed circuit is increased by applying a positive pressure to the closed circuit while introducing liquid into the closed circuit from the outside and also decreasing the pressure by applying a negative pressure to the closed circuit while extracting liquid from the closed circuit to the outside, and the liquid leakage detection is performed in both cases of application of the positive pressure and negative pressure.

27. The method for inspecting liquid leakage of a blood purification apparatus of claim 26, wherein the positive pressure is applied to the closed circuit after the negative pressure has been applied to the closed circuit.

28. The method for inspecting liquid leakage of a blood purification apparatus of claim 15, wherein in the pressure varying step, the pressure in the closed circuit is varied by introducing liquid to the closed circuit through the dialysate introducing line or by discharging liquid from the closed circuit to the dialysate discharging line, and in the liquid leakage detecting step, the liquid leakage in the dialysate introducing line or the dialysate discharging line is detected in addition to liquid leakage in the closed circuit.
